(19) United States
(12) Patent Application Publication
(10) Pub. No.: US 2025/0297232 A1
(43) Pub. Date: Sep. 25, 2025

(54) BACTERIAL MYELOPEROXIDASE-CATALASE AND APPLICATIONS THEREOF

(71) Applicants: UNIVERSITÉ DE BORDEAUX, BORDEAUX (FR); CENTRE NATIONAL DE LA RECHERCHE SCIENTIFIQUE, PARIS (FR)

(72) Inventors: CLAIRE STINES-CHAUMEIL, TALENCE (FR); CLAIRE CÉRÉ, GRANDIGNAN (FR); BRIGITTE DELORD, PESSAC (FR)

(21) Appl. No.: 18/863,011

(22) PCT Filed: Apr. 7, 2023
(86) PCT No.: PCT/EP2023/059317

§ 371 Date: Nov. 5, 2024

(30) Foreign Application Priority Data
May 5, 2022 (EP) 22305670.6

(51) Int. Cl. C12N 9/08; A01N 37/46; C12N 9/04

(52) U.S. Cl. CPC C12N 9/0065; A01N 37/46; C12N 9/0006; C12Y 101/03004; C12Y 111/01006; C12Y 111/02002

(57) ABSTRACT

The present invention relates to the field of enzymology. More particularly, the present invention relates to a novel enzyme having a dual myeloperoxidase-catalase activity, and applications thereof.

### INTRODUCTION

The present invention relates to the field of enzymology. More particularly, the present invention relates to a novel enzyme having a dual myeloperoxidase-catalase activity, and applications thereof.

Heme peroxidases are heme-containing enzymes found in all living organisms, which are capable to catalyze the formation of antimicrobial compounds and to participate in innate immunity. These peroxidases are divided into two main superfamilies: a first family found in plants, fungi and bacteria, which has likely arisen from gene duplication of a single common ancestral gene; and a second family found in mammals, which differs from the first family by its primary and tertiary structures as well as by its prosthetic group. Regardless of their origin, heme peroxidases can display a microbicidal activity thanks to their capacity to halogenate, in presence of hydrogen peroxide, a broad range of organic compounds which can be useful in biomedical, biotechnological or in the food industry. As an illustrative example, heme peroxidases can be a valuable therapy against bacterial infections, such as those that are resistant to antibiotics. That is because heme peroxidases are capable of catalyzing halides or pseudo-halides into (pseudo)hypohalous acids which are known to display potent bactericidal and antiviral activities.

Among heme peroxidases, mammalian peroxidases (MMPs) have been found to play a major role in the destruction of invading pathogens by the innate immune system. To this day, four main four types of mammalian peroxidases have been discovered: the myeloperoxidase (MPO) expressed in neutrophils, the eosinophil peroxidase (EPO) localized in eosinophil granulocytes, the lactoperoxidase (LPO) expressed in mammary, salivary and other mucosal glands, and the thyroid peroxidase (TPO) found in the thyroid gland. While some MMPs can be highly selective for a particular halide, in most cases, such enzymes are capable of oxidizing multiple species of halides: TPO is particularly selective for iodide; human MPO (hMPO) and EPO have the following substrate specificity (from highest to lowest): SCN⁻>I⁻>Br⁻Cl⁻; while LPO has the following substrate specificity (from highest to lowest): SCN⁻>I⁻>>Br. Maturated forms of those enzymes present several post-translational modifications. For example, hMPO possesses light and heavy chains disulfide bridge intra and inter chains glycosylations. The number and the type of covalent bond with heme was in fact identified as a factor influencing substrate specificity. Indeed, the sulfonium bond was found to be responsible for the chlorination activity of hMPO, and to explain the low activity of EPO as well as the lack of activity of the LPO towards this halide. Noticeably, some of these heme peroxidases can display a relatively complex kinetics. For example, depending on the reaction conditions, human MPO can have a halogenating activity, a classic peroxidase activity, a superoxide dismutase activity, a catalase activity, or even an activity similar to cytochrome P450 (by converting O₂ into H₂O), which highlights the very broad potential of this enzyme. From an industrial perspective, the halogenating activity of these enzymes has been the subject of a wide range of applications: porcine MPO has been used in E-101 solution to disinfect human or veterinary injuries (Denys et al., InfectImmun., 2019;87(7):e00261-19); LPO has been identified as a suitable additive for preserving food such as milk, or as an oral disinfectant (WO2008105113A1; WO2011116052); hMPO has been reported to have a virucidal activity against HIV or CMV (Moguilevsky et al., FEBSLett. 1992; 302:209-212; Chochola et al., Antimicrob Agents Chemother, 1994, 38: 969-972; Messaoudi et al., J Med Virol., 2002; 66(2): 218-23), and to be suitable for cleaning contact lenses (WO2003013621A1).

One particular heme peroxidase of bacterial origin, named LsPPOx (Lyngbya sp.), has been extensively studied, and exhibits a strong similarity with LPO. This enzyme can catalyze iodide, bromide, or thiocyanate, but unfortunately does not possess any chlorination activity.

The present Inventors are herein the first to report the discovery, extensive characterization, and recombinant production of a novel bacterial heme peroxidase of about 78 KDa identified in Rhodopirellula baltica. While the whole genome of Rhodopirellula baltica was sequenced nearly 20 years ago, there is no report in the art of the isolation, production, and complete structural and functional characterization of this enzyme. This heme peroxidase is of particularly interest as, despite its low amino sequence identity to hMPO (about 24% sequence identity), it can use a wide range of (pseudo)halides as substrates, including not only iodide, bromide, and thiocyanate but also chloride. Thanks to this unexpected property, this enzyme can display a microbicidal activity, in presence of hydrogen peroxide. Its microbicidal activity can further be potentiated when it is combined with a glucose oxidase which can hydrolyze glucose and accordingly produce hydrogen peroxide. Even more surprisingly, this enzyme exhibits a catalase activity in addition to a peroxidase activity, which makes it suitable for additional applications such as to destroy residual hydrogen peroxide or to detect hydrogen peroxide; it can therefore be qualified as a peroxidase-catalase, more specifically as a myeloperoxidase-catalase, which makes it an atypical enzyme. The Inventors have also discovered a particular composition which stabilizes this bacterial peroxidase-catalase and is thus particularly suited for its preservation and storage.

### SUMMARY OF THE INVENTION

In a first aspect, the invention relates to an isolated polypeptide comprising, or consisting of, an amino acid sequence having at least 95% sequence identity to SEQ ID NO: 1, wherein the polypeptide has a myeloperoxidase-catalase activity.

In a preferred embodiment, the polypeptide of the invention is in the form of a holoenzyme, more preferably is conjugated to, bound to, complexed with, or incorporates heme and optionally calcium.

In a preferred embodiment, the polypeptide of the invention has a myeloperoxidase activity in presence of a halide or pseudohalide and an oxygen donor, at a pH ranging from about 5 to about 8 and/or at a temperature ranging from about 20° C. to about 60° C.

In a preferred embodiment, the halide or pseudohalide is selected from the group consisting of (in order of preference) iodide, thiocyanate, bromide, chloride, and any combinations thereof.

In a preferred embodiment, the polypeptide of the invention has a catalase activity in presence of an oxygen donor, at a pH ranging from about 7 to about 7.5 and/or at a temperature ranging from about 10° C. to about 60° C.

In a preferred embodiment, the oxygen donor is peroxide hydrogen or a source of hydrogen peroxide.

In another aspect, the invention relates to an isolated nucleic acid encoding the polypeptide of the invention, said nucleic acid preferably comprising, or consisting of, a nucleotide sequence having at least 95% sequence identity to SEQ ID NO: 2.

In a further aspect, the invention provides a vector comprising the isolated nucleic acid as disclosed herein.

In another aspect, the invention relates to a host cell comprising the vector of the invention as disclosed herein.

A further aspect pertains to a method for obtaining the isolated polypeptide of the invention, comprising at least the steps of. a) culturing in a medium a host cell of the invention, under conditions suitable for the expression of the polypeptide; and b) recovering said polypeptide.

In a preferred embodiment, the method for obtaining the isolated polypeptide of the invention further comprises step c): solubilizing the recovered polypeptide in a buffer comprising heme and calcium.

In a further aspect, the invention relates to a composition for stabilizing the polypeptide of the invention, said composition comprising, or consisting of, the isolated polypeptide of the invention and a stabilizing agent.

In a preferred embodiment, the stabilizing agent in the composition is Tris buffer.

In a further aspect, the invention relates to a method for stabilizing the isolated polypeptide of the invention, especially when said polypeptide has a myeloperoxidase activity, said method comprising the step of adding a stabilizing agent in an amount sufficient to stabilize the polypeptide described herein to an aqueous solution comprising said polypeptide and optionally freeze-drying the resulting mixture.

Yet, in a further aspect, the invention provides an antimicrobial composition, comprising the isolated polypeptide of the invention, especially when the polypeptide has a myeloperoxidase activity.

In a preferred embodiment, the antimicrobial composition further comprises an oxygen donor, such as hydrogen peroxide or a source of hydrogen peroxide.

In a preferred embodiment, the source of hydrogen peroxide is a peroxide-producing oxidase, preferably selected from the group consisting of a glucose oxidase, a galactose oxidase, a glycollate oxidase, a lactate oxidase, a L-gulunolactone oxidase, a L-2-hydroxyacid oxidase, an aldehyde oxidase, a xhantine oxidase, a D-asparate oxidase, a L-amino acid oxidase, a D-amino acid oxidase, a monoamine oxidase, a pyridoxaminephosphate oxidase, a diamine oxidase, a sulphite oxidase, and any combinations thereof, more preferably is a glucose oxidase.

In a preferred embodiment, when the peroxide-producing oxidase is glucose oxidase, the antimicrobial composition further comprises a glucose or source of glucose and optionally molecular oxygen (O₂).

In a preferred embodiment, the ratio between the isolated polypeptide of the invention and the peroxide-producing oxidase in the antimicrobial composition is such that the polypeptide is present in excess concentration compared to the peroxide-producing oxidase, especially when the peroxide-producing oxidase is a glucose oxidase.

In a preferred embodiment, the concentration ratio between the isolated polypeptide of the invention and the peroxide-producing oxidase in the antimicrobial composition is of at least about 10:1, preferably of at least about 12:1, more preferably of at least about 15:1, especially when the peroxide-producing oxidase is a glucose oxidase.

In a preferred embodiment, the antimicrobial composition further comprises one or more halides or pseudohalides preferably selected from the group consisting of iodide, thiocyanate, bromide, chloride, and any combinations thereof, more preferably thiocyanate, especially when the peroxide-producing oxidase is a glucose oxidase.

In another aspect, the invention relates to a medical device treated with or coated with the isolated polypeptide, nucleic acid or composition of the invention.

In a further aspect, the invention pertains to an in vitro use of the isolated polypeptide, or nucleic acid or composition of the invention, for halogenating a non-halogenated organic compound, especially when the polypeptide has a myeloperoxidase activity.

In another aspect, the invention relates to an in vitro or ex vivo use of the isolated polypeptide or nucleic acid or composition of the invention, for killing or inhibiting the growth of microorganisms, especially when the polypeptide has a myeloperoxidase activity.

In a further aspect, the invention relates to the isolated polypeptide or nucleic acid or composition of the invention, for use as a medicament, preferably for the treatment of a microbial infection, especially when said polypeptide has a myeloperoxidase activity.

Yet, in another aspect, the invention further relates to (i) the isolated polypeptide or nucleic acid of the invention and (ii) an oxygen donor such as hydrogen peroxide or a source of hydrogen peroxide, as a combined preparation for simultaneous, separate or sequential use as a medicament, preferably for the treatment of a microbial infection, especially when said polypeptide has a myeloperoxidase activity.

Still, in a further aspect, the invention provides an in vitro or ex vivo use of the isolated polypeptide, nucleic acid or composition of the invention, for converting hydrogen peroxide into oxygen and water, especially when the polypeptide has a catalase activity.

### LEGENDS TO THE FIGURES

FIG. 1. Recapitulative scheme of activities displayed by the RbMPO enzyme according to the invention.

FIG. 2. Sequence alignment of four mammalian peroxidases (hMPO, LPO, EPO, TPO) with the wt RbMPO enzyme (SEQ ID NO: 1) according to the invention. a) Essential amino acids of the distal heme cavity site. b) Essential amino acids of the Ca²⁺ binding site. c) Essential amino acids of the proximal heme cavity site. *hMPO amino acid numbering corresponds to the numbering in the PDB 1CXP. The three arrows correspond to the amino acid residues involved in covalent binding to heme. Dashed arrow corresponds to the sulfonium linkage only present in hMPO.

FIG. 3. Superposition of the 3D structures of hMPO (1CXP: PDB code) and the 3D structure prediction of wt RbMPO (SEQ ID NO: 1) obtained via Phyre 2 and from bovin lactoperoxidase. The amino acids of the active site that was mutated are in bold italic for hMPO and in bold underlined for those corresponding of RbMPO. This figure was made on Pymol (version 1.7.4.4.5).

FIG. 4. Superposition of UV-visible spectrum of wt RbMPO and RbMPOs displaying mutations in the active site. Origin 2019, version 9.6.0.172.

FIG. 5. Reconstitution efficiency of apo wt and mutated RbMPOs and site titration. (A) Fluorescence emission spectra (λₑₓ=297 nm) of apo wt RbMPO before and after addition of various heme concentration. (B) Time-course of fluorescence quenching of tryptophan emission at 334 nm (λₑₓ=297 nm) during the conversion of apo wt RbMPO to the holo form after addition of various heme concentrations. (C) Titration plot of apo wt RbMPO by heme from screening effect obtained from the plot (B) at T0+ as a function of [heme]/[apo wt RbMPO]with aspecific correction. (D) Histograms of heme reconstitution of apo wt and mutated RbMPOs. 1 μM of enzyme in sodium phosphate 50 mM pH7.5 was used for all assays, which were performed at 25° C. Origin 2019, version 9.6.0.172.

FIG. 6. Effect of ABAH on the chlorination activity of wt RbMPO. Fixed concentration of enzyme and substrates were used: 1 M of wt RbMPO, 500 mM of NaCl, 500 M of H₂O₂ and 10 μM of APF. Each measurement was performed in triplicate at 37° C., and in 50 mM NaPi buffer pH7.5. Origin 2019, version 9.6.0.172.

FIG. 7. Effect of increasing ABAH concentration on tryptophan fluorescence emission of wt and mutated RbMPOs. (A) Fluorescence emission spectra (λₑₓ=297 nm) of holo wt RbMPO before and after addition of various heme concentrations. (B) Temporal evolution of the fluorescence quenching of tryptophan emission at 334 nm (λₑₓ=297 nm) during the conversion of holo wt RbMPO to its apo of form after addition of various heme concentrations. (C) Histogram representation for heme rearrangement efficiency for wt and mutated RbMPOs. 1 μM in site of RbMPOs was used in a 50 mM pH7.5 sodium phosphate buffer at 25° C. Origin 2019, version 9.6.0.172.

FIG. 8. Catalytic efficiency of wt RbMPO and mutated RbMPOs for (pseudo-halides). (A) Histogram representation of the catalytic efficiency for SCN⁻. (B) Histogram representation of the catalytic efficiency for Br⁻. Origin 2019, version 9.6.0.172.

FIG. 9. Kinetic parameters of wt RbMPO under pre-steady-state conditions. (A) Effect of H₂O₂ concentrations on the formation of compound I. (B) Effect of (pseudo)-halides on the return to the native state of wt RbMPO. (B.1) Return to the native state in presence of Cl⁻ or Br⁻; (B.2) Return to the native state in presence of SCN⁻ or I⁻. Temperature was set at 37° C. and sample dilution was made in a 50 mM pH7.5 sodium phosphate buffer. Each condition had a minimum average of 5 points. Origin 2019, version 9.6.0.172.

FIG. 10. Determination of kinetics parameters of chlorination activity of wt and mutated RbMPOs. (A) The NaCl range was as follow: 0, 10, 20, 50, 100, 250, 500, 750 and 1000 mM at fixed concentration of RbMPOs (1 μM), H₂O₂(79 μM) and APF (10 μM). (B) Histogram representation of the catalytic efficiency of RbMPOs for chloride. (C) The H₂O₂ range was as follow: 0, 10, 20, 50, 79, 100, 250, 500, 750 μM, 1, 2, 4, 6, 8, 10, 20, 40, 60, 80, 100, 200 and 500 mM at fixed concentration of RbMPOs (1 μM), NaCl (500 mM) and APF (10 μM). (D) Histogram representation of the catalytic efficiency of RbMPOs for H₂O₂. For all these experiments, each measurement was made in triplicate at 37° C. in a 50 mM NaPi pH7.5 buffer. Origin 2019, version 9.6.0.172.

FIG. 11. Catalase activity of wt RbMPO (A) As a function of pH. Each curve corresponds to an H₂O₂ range with fixed concentration of the enzyme (2 μM) at different pH values. Temperature was set at 37° C. and sample dilution was made in a 50 mM pH7.5 sodium phosphate buffer. Each condition had a minimum average of 5 points. (B) As a function of temperature. Each histogram corresponds to a temperature between 1° and 60° C. with fixed concentration of the enzyme (300 nM) in sodium phosphate buffer at pH 7.5 and 500 μM H₂O₂.

FIG. 12. Peroxidase activity of wt RbMPO. Measurements were made at 430 nm and 37° C. in a 50 mM NaPi buffer at pH5.5. Fixed concentration of enzyme (0.5 nM) and H₂O₂(79 μM) were used with an ABTS range. Each condition was an average of 3 wells. Origin 2019, version 9.6.0.172.

FIG. 13. Myeloperoxidase activity of wt RbMPO after storage of the enzyme using different buffers. Activity of wt RbMPO stored in: a Tris 50 mM buffer pH 7.5 at 4° C. (A) and 37° C. (B), for up to 30 days; or in a 50 mM NaPi buffer pH 7.5, at 4° C. (C) and 37° C. (D), for up to 60 days; or in a PBS buffer pH6.6 for up to 60 minutes (E). The myeloperoxidase activity was measured at 525 nm after excitation at 485 nm at 37° C., using fixed concentration of enzyme, NaCl and H₂O₂ in presence of AFP.

FIG. 14. Bactericidal activity of wt RbMPO. (A to C) A₆₂₀ₘ measurement in a 16h kinetic in presence of three concentrations of GOx_{aspniger} ((A) 10, (B) 25 or (C) 30 nM), a fixed concentration of SCN⁻ (25 mM) and a concentration range of wt RbMPO (CS: coupled system “GOx_{aspniger} and wt RbMPO”) from 6.25 to 2000 nM. (D) A₆₂₀ₙₘ at 410 min for each tested condition in presence of fixed concentration of SCN⁻ and a range of GOx_{aspniger} and wt RbMPO. (E) A₆₂₀ₙₘ at 820 min for each tested condition, in presence of fixed concentration of SCN⁻ and a range of GOx_{aspniger} and wt RbMPO. (F) Histogram summarizing the different conditions tested in the presence of 1.10⁶ CFU/mL (noted B⁻ on the x-axis), and in contact for 1 h with the enzymatic solution at pH7.4. The CFU/mL defined on the y-axis (in log 10) corresponds to the number of bacteria surviving after one night at 37° C. in the incubator. The 2nd ordinate-axis corresponds to the enzyme concentration (wt RbMPO alone, GOx_{aspniger} alone or coupled system) present in the incubation solution. For all experiments, the bactericidal activity of wt RbMPO was tested in presence of SCN⁻ and glucose (14 mM), and measurements were made at 37° C. and each condition had a minimum average n=3 except for FIG. 14F.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, nomenclatures used herein, and techniques of molecular and cellular therapy are those well-known and commonly used in the art.

The present invention may be understood more readily by reference to the following detailed description, included preferred embodiments of the invention, and examples included herein.

The present invention provides a novel bacterial enzyme isolated from Rhodopirellula baltica, with a dual activity, namely a myeloperoxidase activity and a catalase activity, and functional variants thereof. Key amino acid residues involved in the biological activity of this novel enzyme have notably been identified by the Inventors.

Accordingly, in a first aspect, the present invention relates to an isolated polypeptide comprising, or consisting of, an amino acid sequence having at least 95% sequence identity to SEQ ID NO: 1, wherein the polypeptide has a myeloperoxidase-catalase activity.

As used herein, the terms “polypeptide” and “protein” are used interchangeably to refer to a precise succession of amino acid residues, also referred as amino acid sequence. As such, these terms include polypeptides of any size, preferably of at least 500, 550, 600, 650, 660, 670, 680, 690 or 700 amino acids, and/or polypeptides that have undergone post-translational modifications.

By “isolated”, it is meant herein free or separated from its natural environment, or at least some components thereof. For example, “isolated” can mean that at least one order of magnitude of purification is achieved, preferably two or three orders of magnitude, and most preferably four to five orders of magnitude of purification of the starting material or natural material. In other words, as used herein “isolated” does not necessarily mean that the material of interest is 100% purified, as long as it does not interfere with the biological activity.

In the context of the present invention, the polypeptide is preferably isolated from Rhodopirellula baltica.

The terms “activity”, “function”, “biological activity”, and “biological function” are equivalent and have to be understood as well known in the art. Preferably, such an activity is enzymatic. That is, in the context of the invention, the activity exhibited by the isolated polypeptide of the invention is one of a myeloperoxidase and/or of a catalase, referred herein as a myeloperoxidase-catalase activity.

A “myeloperoxidase activity” is typically characterized, as explained above, by the oxidation of halides or pseudo-halides into (pseudo)hypohalous acids, in the presence of hydrogen peroxide, according to the following reaction:

H₂O₂+X⁻+H⁺→H₂O+HOX

wherein X⁻ represents a halide or a pseudohalide.

The term “halide” refers to an ion of a halogen, and includes herein chloride (Cl⁻), bromide (Br⁻), or iodide (I⁻), and any combination thereof. The term “pseudohalide” refers to a polyatomic anion resembling the halides in their acid-case and redox chemistry, and includes herein thiocyanate (SCN). Halides and pseudohalides are referred herein globally as (pseudo)halides.

A myeloperoxidase activity can be detected according to the protocols described in sections 1.7 to 1.11 of the Examples below, and/or measured according to the protocols described by Tenovuo et al. (Biochim Biophys Acta, 1986; 870(3): 377-84), Auer et al. (J Biol Chem., 2013; 288(38): 27181-27199) and/or Flemmig et al. (J Biol Chem., 2012; 287(33): 27913-23).

A “catalase activity” is typically characterized by the catalyzation of hydrogen peroxide into water and oxygen, according to the following reaction:

2H₂O₂→2H₂O+O₂

A catalase activity can be detected according to the protocol described in section 1.12 of the Examples below, and/or measured according to the protocol described by Hadwan et al. (Journal of Clinical and Diagnostic Research, 2018; 12(9):13-16).

As demonstrated in the Examples below, in order to be enzymatically active, the polypeptide of the invention is in the form of a holoenzyme (or haloenzyme).

In other words, in order to exhibit a myeloperoxidase-catalase activity, the polypeptide of the invention is conjugated to, bound to, complexed with, or incorporates a cofactor and optionally metal ions. A “cofactor” refers herein to a non-protein molecule that is required for some enzymes to be catalytically active.

More precisely, the polypeptide of the invention is conjugated to, bound to, complexed with, or incorporates heme as cofactor and optionally calcium as metal ions. In short, the polypeptide of the invention is a heme-containing polypeptide. The term “heme”, “heme cofactor”, or “heme moiety” is meant herein to refer to an iron-containing compound of the porphyrin class, whether iron is in a ferrous (Fe²⁺) or in a ferric (Fe³⁻) state. In a preferred embodiment, the iron is in a ferrous state (Fe²⁺).

When the polypeptide of the invention is in the form of a holoenzyme, it can form oligomers. For example, the polypeptide can be a monomer, or a multimer. More preferably, the polypeptide of the invention is a monomer.

An “oligomer” or “oligomeric state” refers herein to the structural unit(s) that makes up an oligomeric polypeptide. The number (n) of these structural units, also known as the degree of oligomerization, can be equal or superior to 1 (n≥1). When n is superior to 1, the structural unit(s) are typically linked together either covalently or non-covalently. n is generally less than one hundred, usually less than thirty. An oligomer with n=1 is known as a monomer or a single unit, while an oligomer with n≥1 can be referred as a multimer or a multi-unit. A monomer or single unit is typically made herein of one polypeptide (or polypeptide chain), while a multimer or a multi-unit is typically made of at least two polypeptides (or polypeptide chains). Oligomers of increasing length are called dimer (n=2), trimer (n=3), tetramer (n=4), pentamer (n=5), hexamer (n=6), heptamer (n=7), octamer (n=2), nonamer (n=9), decamer (n=10), etc.

The present invention embraces an isolated polypeptide comprising, or consisting of, the amino acid sequence SEQ ID NO: 1, as described above, as well as variants thereof as long as these remain functional and have less than 5% sequence variation compared to SEQ ID NO: 1, or in other words, have at least 95% sequence identity to SEQ ID NO: 1.

By “variant” or “functional variant” of a polypeptide (or of the nucleic acid that encodes said polypeptide) of interest, it is meant a polypeptide (or nucleic acid) that structurally differ from the amino acid (or nucleotide) sequence of reference but that generally retains the same or substantially the same essential biological activity as the polypeptide (or nucleic acid) of reference. Variants can typically comprise e.g. silent mutations, conservative mutations, or minor deletions of genetic material, which do not impact or substantially impact the biological activity of the biological material of reference.

“Sequence identity” between amino acid or nucleic acid sequences can be determined by comparing a position in each of the sequences which may be aligned for the purposes of comparison. When a position in the compared sequences is occupied by the same amino acid or nucleotide, then the sequences are identical at that position. A degree of identity between amino acid sequences is a function of the number of identical amino acid sequences that are shared between these sequences. A degree of sequence identity between nucleic acids is a function of the number of identical nucleotides at positions shared by these sequences.

To determine the “percentage of sequence identity” between two amino acid sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percentage (%) of identity between the two sequences is a function of the number of identical positions shared by the sequences. Hence, the percentage of identity can be calculated by multiplying the number of identical positions by 100 and dividing by the length of the aligned region (overlapping positions), including gaps (only internal gaps, not the gaps at the sequence ends). In this comparison, the sequences can be of the same length, or may be of different lengths. Identity scoring only counts perfect matches, and does not consider the degree of similarity of amino acids to one another. The percentage of identity is herein calculated over the entire length of the sequence of reference.

Optimal alignment of sequences may be herein preferably conducted by a global homology alignment algorithm, such as by the algorithm described by Needleman and Wunsch (Journal of Molecular Biology, 1970, 48(3): 443-53), by computerized implementations of this algorithm, or by visual inspection. A global homology alignment is particularly preferred if the alignment is performed using sequences of the same or similar length.

In a preferred embodiment, functional variants of the invention have less than 5% amino acid (or nucleotide) variation as compared to SEQ ID NO: 1 (or SEQ ID NO: 2), preferably less than 4% or 3%, more preferably less than 2% or 1% amino acid (or nucleotide) variation as compared thereto. In other words, preferred functional variants of the invention have at least 95% sequence identity to SEQ ID NO: 1 (or SEQ ID NO: 2), preferably at least 96% or 97%, more preferably at least 98% or 99% sequence identity thereto.

In a preferred embodiment, functional variants of the invention may comprise conservative substitutions, especially in non-critical residues or in non-critical regions.

“Conservative substitution” as used herein denotes the replacement of an amino acid (or corresponding codon) residue by another, without altering the overall conformation and function of the polypeptide (or corresponding nucleic acid) of reference, including, but not limited to, replacement of an amino acid (or corresponding codon) with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties. Examples of conservative substitutions are set out in the Table 1 below.

TABLE 1
Conservative substitutions I

| Side chain characteristic | Amino Acid |
|---|---|
| Non-polar | G, A, P, I, L, V |
| Polar uncharged | C, S, T, M, N, Q |
| Polar-charged | D, E, K, R |
| Aromatic | H, F, W, Y |
| Other | N, Q, D, E |

Alternatively, conservative amino acids can be grouped as described in Lehninger, 1975, as set out in Table 2 below.

TABLE 2
Conservative substitutions II

| | |
|---|---|
| Side chain characteristic | Amino Acid |

Non-polar (hydrophobic)

| | |
|---|---|
| Aliphatic | A, L, I, V, P |
| Aromatic | F, W |
| Sulfur-containing | M |
| Borderline | G |

Polar uncharged

| | |
|---|---|
| Hydroxyl | S, T, Y |
| Amides | N, Q |
| Sulfhydryl | C |
| Borderline | G |

Polar-charged

| | |
|---|---|
| Positively charged (basic) | K, R, H |
| Negatively charged (acidic) | D, E |

As a further alternative, exemplary conservative substitutions are set out in Table 3 below.

TABLE 3
Conservative substitutions III

| Original amino | |
|---|---|
| acid residue | Amino acid substitution |
| Ala (A) | Val (V), Leu (L), Ile (I) |
| Arg (R) | Lys (K), Gln (Q), Asn (N) |
| Asn (N) | Gln (Q), His (H), Lys(K), Arg (R) |
| Asp (D) | Glu (E) |
| Cys (C) | Ser (S) |
| Gln (Q) | Asn (N) |
| Glu (E) | Asp (D) |
| His (H) | Asn (N), Gln (Q), Lys (K), Arg (R) |
| Ile (I) | Leu (L), Val (V), Met (M), Ala (A), Phe (F) |
| Leu (L) | Ile (I), Val (V), Met (M), Ala (A), Phe (F) |
| Lys (K) | Arg (R), Gln (Q), Asn (N) |
| Met (M) | Leu (L), Phe (F), Ile (I) |
| Phe (F) | Leu (L), Val (V), Ile (I), Ala (A) |
| Pro (P) | Gly (G) |
| Ser (S) | Thr (S) |
| Thr (T) | Ser (S) |
| Trp (W) | Tyr (T) |
| Tyr (Y) | Trp (W), Phe (F), Thr (T), Ser (S) |
| Val (V) | Ile (I), Leu (L), Met (M), Phe (F), Ala (A) |

Amino acid residues that are critical for the biological activity of the polypeptide of the invention have been identified by the Inventors; these include amino acids at positions 199, 202, 203, 316, 317, and 407 by reference to the numbering of the amino acid sequence SEQ ID NO: 1. These amino acids are more particularly Q199, D202, H203, E316, N317, and H407.

Accordingly, in a preferred embodiment, functional variants of the invention have less than 5% amino acid (or nucleotide) variation as compared to SEQ ID NO: 1 (or SEQ ID NO: 2), preferably less than 4% or 3%, more preferably less than 2% or 1% amino acid (or nucleotide) variation as compared thereto, with the proviso that the following amino acid residues (or corresponding codons) are conserved: Q199, D202, H203, E316, N317, and H407.

In other words, functional variants of the invention preferably have at least 95% sequence identity to SEQ ID NO: 1 (or SEQ ID NO: 2), preferably at least 96% or 97%, more preferably at least 98% or 99% sequence identity thereto, with the proviso that the following amino acid residues (or corresponding codons) are conserved: Q199, D202, H203, E316, N317, and H407.

Methods for preparing the polypeptide of the invention are as described below.

As explained above, the Inventors have herein characterized the enzymatic activity of the polypeptide of the invention, and identified that the latter can surprisingly behave as a myeloperoxidase or as a catalase depending on the incubating conditions, such as the substrate, the pH and/or the temperature.

More precisely, the polypeptide of the invention has preferably a myeloperoxidase activity in presence of a halide or pseudohalide and an oxygen donor, at a pH ranging from about 5 to about 8 and/or at a temperature ranging from about 20° C. to about 60° C.

By “about”, it is meant that the measured value may vary within a certain range depending on the margin of error of the method or device used to evaluate the parameter of interest, such as, without limitation, pH, temperature, or concentration. In the context of the present invention, it preferably means that the measured value may be 10% away, advantageously 5%, 4%, 3%, 2%, or 1% away, from the given numerical value and that the recited range includes both endpoints.

The Inventors have also discovered that, when the polypeptide of the invention behaves as a myeloperoxidase, said enzyme can surprisingly catalyze a wide range of halides and pseudohalides.

In particular, the halide or pseudohalide is preferably selected from the group consisting of (in order of preference) iodide, thiocyanate, bromide, chloride, and any combinations thereof

In a preferred embodiment, the polypeptide of the invention has a myeloperoxidase activity in presence of a halide or pseudohalide and an oxygen donor, at a pH ranging from about 5 to about 8, such as at a pH of about 5, about 5.5, about 6, about 6.5, about 7, about 7.5 or about 8.

For example, when the pseudohalide is thiocyanate, the polypeptide of the invention has a myeloperoxidase activity in presence of said pseudohalide and an oxygen donor, preferably at a pH ranging from about 5 to about 8, more preferably at a pH ranging from about 6 to about 8, such as at a pH of about 6, about 6.5, about 7, about 7.5 or about 8, more preferably at a pH of about 7.

As another example, when the halide is bromide, the polypeptide of the invention has a myeloperoxidase activity in presence of said halide and an oxygen donor, preferably at a pH ranging from about 6 to about 7, such as at a pH of about 6, about 6.5 or about 7, more preferably at a pH of about 6.

Yet, as another example, when the halide is chloride, the polypeptide of the invention has a myeloperoxidase activity in presence of said halide and an oxygen donor, preferably at a pH ranging from about 5 to about 7, such as at a pH of about 5, about 5.5. about 6, about 6.5 or about 7, more preferably at a pH of about 7.

Still, as another example, when the halide is iodide, the polypeptide of the invention has a myeloperoxidase activity in presence of said halide and an oxygen donor, preferably at a pH ranging from about 7 to about 8, such as at a pH of about 7, about 7.5 or about 8, more preferably at a pH of about 7.5.

Yet, in a preferred embodiment, the polypeptide of the invention has a myeloperoxidase activity in presence of a halide or pseudohalide and an oxygen donor, at a temperature ranging from about 20° C. to about 60° C., such as at a temperature of about 20° C., about 25° C., about 30° C., about 35° C., about 40° C., about 45° C., about 50° C., about 55° C., or about 60° C.

For example, when the halide or pseudohalide is iodide, thiocyanate, bromide, chloride, or any combinations thereof, the polypeptide of the invention has a myeloperoxidase activity in presence of said (pseudo)halide and an oxygen donor, preferably at a temperature ranging from about 20° C. to about 60° C., such as at a temperature of about 20° C., about 25° C., about 30° C., about 35° C., about 40° C., about 45° C., about 50° C., about 55° C., or about 60° C., more preferably at a temperature of about 35° C. (such as 37° C.) or about 60° C.

In another preferred embodiment, the polypeptide of the invention has a catalase activity in presence of an oxygen donor, at a pH ranging from about 7 to about 7.5 and/or at a temperature ranging from about 10° C. to about 60° C.

In a preferred embodiment, the polypeptide of the invention has a catalase activity in presence of an oxygen donor, at a pH ranging from about 7 to about 7.5, such as at pH of about 7, about 7.25, or about 7.5, preferably at a pH of about 7.5.

In a preferred embodiment, the polypeptide of the invention has a catalase activity in presence of an oxygen donor, at a temperature ranging from about 10° C. to about 60° C., such as at a temperature of about 10° C., about 15° C., about 20° C., about 25° C., about 30° C., about 35° C., about 40° C., about 45° C., about 50° C., about 55° C., or about 60° C., preferably at a temperature ranging from about 20° C. to about 40° C., such as at a temperature of about 20° C., about 25° C., about 30° C., about 35° C., or about 40° C., more preferably at a temperature of about 35° C. (such as 37° C.).

Whether the polypeptide of the invention has a myeloperoxidase or catalase activity, a particularly preferred oxygen donor according to the invention is peroxide hydrogen or a source of hydrogen peroxide. Particularly preferred sources of hydrogen peroxide are further detailed below.

The isolated polypeptide according to the invention is preferably encoded by a nucleic acid, such as the one isolated from Rhodopirellula baltica, or functional variants thereof.

By “nucleic acid” or “nucleotide sequence”, it is meant herein a precise succession of natural nucleotides (namely, A, T, G, C and U) or non-natural nucleotides. These terms encompass a single-stranded or double-stranded DNA, as well as the transcription product of said DNA, such as an RNA.

Accordingly, in a further aspect, the present invention pertains to an isolated nucleic acid encoding the polypeptide as described herein, said nucleic acid preferably comprising, or consisting of, a nucleotide sequence having at least 95% sequence identity to SEQ ID NO: 2.

In other words, the present invention embraces an isolated nucleic acid encoding the polypeptide of the invention, wherein the nucleic acid comprises, or consists of, the nucleotide sequence SEQ ID NO: 2, as well as variants thereof as long as these remain functional and have less than 5% sequence variation compared to SEQ ID NO: 2, i.e. have at least 95% sequence identity to SEQ ID NO: 2.

In a preferred embodiment, functional variants of the invention have less than 5% nucleotide (or amino acid) variation as compared to SEQ ID NO: 2 (or SEQ ID NO: 1), preferably less than 4% or 3%, more preferably less than 2% or 1% nucleotide (or amino acid) variation as compared thereto. In other words, preferred functional variants of the invention have at least 95% sequence identity to SEQ ID NO: 2 (or SEQ ID NO: 1), preferably at least 96% or 97%, more preferably at least 98% or 99% sequence identity thereto.

In a preferred embodiment, functional variants of the invention may comprise conservative substitutions, especially in non-critical codons or in non-critical regions. Preferred conservative substitutions are as described above.

The nucleic of the invention can be prepared by methods well-known in the art, including, but not limited to, any synthetic and/or recombinant method.

In the context of the present invention, the nucleic acid is preferably isolated from Rhodopirellula baltica.

The isolated nucleic acid according to the invention can advantageously be comprised in a vector in order to amplify this nucleic acid, or to express the polypeptide of the invention in a host cell.

It is thus a further aspect of the invention to provide a vector comprising the nucleic acid as disclosed herein.

Said vector can advantageously be comprised in a host cell, such as a prokaryotic or a eukaryotic cell. Accordingly, the vector can be a prokaryotic or eukaryotic vector.

The invention thus also relates to a host cell comprising the vector of the invention.

The term “vector” generally refers to a tool useful for performing procedures of molecular biology and genetic recombination. Such tool is commonly used and very well known in the art. This term encompasses vectors capable of replication in order to amplify a nucleic acid of interest (i.e. a cloning vector), or to express the polypeptide encoded by said nucleic acid in a host cell (i.e. an expression vector). These types of vectors are publicly available and include, without limitation, plasmids, cosmids, YACS, BACS, viral vectors (adenovirus, AAV, retrovirus such as lentivirus, EBV episome, etc.), and phage vectors. The vector is herein said to be recombinant in that it is not found in nature combined to the nucleic acid of the invention (i.e. it is not naturally-occurring).

Methods for inserting a nucleic acid into a vector are known to the skilled practitioner. Generally, a nucleic acid can be inserted into one or more restriction endonuclease site(s) using techniques well-known in the art (see, for example, the techniques described by Sambrook et al. in Molecular Cloning: A Laboratory Manual, 4^{th} edition, 2012). Nucleotide sequences allowing the transcription of said nucleic acid, the expression and/or purification of the protein encoded by said nucleic acid are preferably also contained in the vector. These sequences include, generally and without limitation, at least one sequence selected from one or more signal peptide sequence(s), an origin of replication, one or more gene(s) marker(s) selection, an enhancer element, a promoter, a transcription terminator, and possibly a sequence allowing purification of a protein. The insertion of such sequences in said vector can be done via standard ligation techniques known to those skilled in the art, such as mentioned above. It is additionally known to those skilled in the art that these nucleotide sequences can be selected based on the host cell in which the vector is intended to replicate, and/or in which the polypeptide encoded by the nucleic acid is intended to be expressed.

For example, depending on the selected replication origin, the vector may replicate in one or more host cells: the origin of replication of plasmid pBR322 is typically adapted to most Gram-negative bacteria, that of plasmid 2 is generally specific to yeast, and various origins of viral replication (SV40, polyoma, adenovirus, VSV or BPV) are particularly useful for cloning vectors in mammalian cells.

As another example, depending on the selected promoter, the isolated nucleic acid may be transcribed and the corresponding polypeptide expressed in one or more host cells: promoters T7, Lac, trp, tac, XPL are typically specific for E. coli bacteria; promoters PHO5, GAP, TPI1, ADH are generally adapted to yeast; promoters of polyhedrin and P10 and their equivalents are conventionally used in insect cells; finally, promoter CMV, MT1, SV40, SRα, retroviral and gene promoters of a heat shock protein are particularly adapted to mammalian cells.

In a preferred embodiment, the vector of the invention comprises the T7 promoter.

Non-exhaustive examples of selection marker genes typically contained in vectors are genes conferring resistance to an antibiotic or toxin (e.g, ampicillin, neomycin, zeocin, hygromycin, kanamycin, tetracycline, chloramphenicol, or combinations thereof), and genes allowing the compensation of an auxotrophic deficiency (e.g. the gene coding for dihydrolofate reductase DHFR allowing resistance to methotrexate, or still the TPI gene of S.pombe).

In a preferred embodiment, the vector of the invention comprises the selection markers ampicillin and chloramphenicol.

Non-exhaustive examples of nucleotide sequences that allow the purification of a polypeptide are the Histidine sequence (Histidine Tag or Hisx6), the FLAG sequence and the GST sequence. A cleavage sequence of a protease, such as VTE, may further be present in order to subsequently delete the purification sequence.

In a preferred embodiment, the vector of the invention comprises the Histidine sequence such as Hisx6.

Non-exhaustive examples of prokaryotic vectors are: pET (Novagen), pQE70, pQE60, pQE-9 (Qiagen), pbs, pDIO, phagescript, psiX174, pbluescript SK, pbsks, pNH8A, pNH16A, pNH18A, pNH46A (Stratagene); ptrc99a, pKK223-3, pKK233-3, pDR540, pBR322, and pRIT5 (Pharmacia).

Non-exhaustive examples of eukaryotic vectors are: pWLNEO, pSV2CAT, pPICZ, pcDNA3.1 (+) Hyg (Invitrogen), pOG44, pXT1, pSG (Stratagene); pSVK3, pBPV, pCI-neo (Stratagene), pMSG, pSVL (Pharmacia); and pQE-30 (QLAexpress).

In a preferred embodiment, the vector of the invention is a prokaryotic vector, preferably the pET vector, such as the pET21a vector.

As used herein, the terms “host cell”, “cell” and “cell line” can be used interchangeably, and refer to a prokaryotic or eukaryotic cell in which the vector of the invention can be introduced, such as to amplify the isolated nucleic acid as described above, and/or to express the polypeptide encoded by said nucleic acid. To this end, a host cell may be “transfected” or “transformed” by a process known in the art by which said vector is transferred or introduced into the host cell. Examples of such methods include, without limitation, electroporation, lipofection, calcium phosphate transfection, transfection using DEAE dextran, microinjection, and biolistics.

The choice of the host cell typically depends on the selected use, namely the cloning of the nucleic acid or the expression of the polypeptide encoded by said nucleic acid. The skilled person will be able to choose the appropriate host cell among the many cell lines that are publicly available, notably via the American Type Culture Collection (ATCC).

Examples of prokaryotic cells include, without limitation, bacteria such as Gram-negative bacteria of the genus Escherichia (e.g. E. coli BL21, C41, RR1, LE392, B, X1776, W3110, DH5 alpha, JM109, KC8), Serratia Pseudomonas, Erwinia Methylobacterium, Rhodobacter, Salmonella and Zymomonas, and Gram positive bacteria of the genus Corynebacterium, Brevibacterium, Bacillus, Arthrobacter, and Streptomyces.

Examples of eukaryotic cells include, without limitation, cells isolated from fungi, plants, and animals. Such cells notably include, without limitation, yeasts such as those of the genus Saccharomyces; cells from a fungus such as those of the genus Aspergillus, Neurospora, Fusarium or Trichoderma; animal cells such as HEK293 cells, NIH3T3, Jurkat, MEF, Vero, HeLa, CHO, W138, BHK, COS, COS-7, MDCK, C127, Saos, PC12, HKG; and insect cells such as Sf9, Sf21, Hi Five™ or of Bombyx mori.

In a preferred embodiment, the host cell of the invention is a prokaryotic cell, preferably of the genus Escherichia, more preferably E. coli such as E. coli BL21 (e.g. BL21 DE3 pLysS).

Polypeptides having a myeloperoxidase and/or catalase activity are typically difficult to extract from their native source, which can lead to inadequate yields and to a mixture of enzymes differing in glycosylation, thereby limiting their subsequent applications.

The present Inventors are herein the first to demonstrate the recombinant production of the polypeptide of the invention, at a high yield, while still allowing a proper refolding of the polypeptide.

According to a further aspect, the invention relates to a method for obtaining the isolated polypeptide of the invention, comprising at least the steps of:
a) culturing in a medium a host cell of the invention, under conditions suitable for the expression of the polypeptide; and
b) recovering the polypeptide.

In a further aspect, the invention is related to a method for obtaining the isolated polypeptide of the invention, comprising at least the steps of:
a′) cloning an isolated nucleic acid as described above, into an expression vector;
b′) transforming a host cell with said expression vector; and
c′) expressing said isolated nucleic acid from the host cell obtained in step b′), so as to obtain said polypeptide.

To do so, the host cell is typically cultured in a suitable culture medium under conditions allowing the expression of the nucleic acid, and thus of the polypeptide.

The vector and/or host cell used in said method(s) are preferably as described above.

In a preferred embodiment, the vector is a prokaryotic vector, preferably the pET vector, such as the pET21a vector, and/or the host cell is a prokaryotic cell, preferably of the genus Escherichia, more preferably E. coli such as E. coli BL21 (e.g. BL21 DE3 pLysS).

In particular, the polypeptide of step b) or c′) can be obtained by recovering the polypeptide from the host cells if said polypeptide is expressed intracellularly, and/or from the culture medium in which the host cells are cultured if said polypeptide is expressed extracellularly.

The polypeptide recovered in step b) or obtained in step c′) can be advantageously purified, in a further step of said method. Preferably, said purification step allows the obtention of a 100%-purified or almost 100%-purified polypeptide.

The skilled person in the art may use any conventional method allowing the isolation and/or purification of said polypeptide. For example, if the polypeptide was expressed in a dissolved form in host cells, the latter can be recovered by centrifugation and suspended in a buffer, then a cell-free extract can be obtained by destroying the cells through e.g. an ultrasonic homogenizer (sonication) or a cell disruptor optionally combined with an urea treatment. From the supernatant obtained by centrifugation of this extract, a purified sample can be obtained using a conventional method or combination of conventional methods to isolate and purify the polypeptide of the invention. These methods include, without limitation, solvent extraction, salting out with ammonium sulphate, desalting, precipitation with dialysis, filtration, ultrafiltration, organic solvent, preparative electrophoresis, isoelectric focusing, various chromatographic methods such as ion exchange chromatography (anionic, using for example a resin such as diethylaminoethyl (DEAE) Sepharose; or cationic, by using for example a resin such as S-Sepharose (Pharmacia), hydrophobic chromatography (using for example a resin such as butyl sepharose or phenyl sepharose), affinity chromatography using antibodies, adsorption chromatography, chromatofocusing, high performance liquid chromatography (HPLC) and reversed phase HPLC, and any combinations thereof.

Moreover, if a nucleotide sequence allowing purification of the polypeptide, such as a histidine tag, is present in the vector or in the nucleic acid of the invention, as described above, the polypeptide thereby produced can be recovered by cleavage of said sequence through a specific protease (e.g. thrombin, trypsin, protease TEV, etc).

In the case where a chromatographic method is used for purification, one or more substeps can be performed and include, without limitation, the binding of the recovered or obtained polypeptide on a solid support, such as a chromatography column, a washing step, and an elution step. Said substeps can be repeated as many times as necessary in order to achieve the desired degree of purification of the polypeptide, preferably a 100% or almost 100% purification of the isolated polypeptide.

According to a preferred embodiment, the polypeptide recovered in step c) or obtained in step d′) can be solubilized in a further step of said method, preferably in a suitable buffer. More preferably, the solubilization step allows the obtention of a fully folded and functional polypeptide. The solubilization buffer can notably contain calcium such as CaCl₂, so as to facilitate the formation of a protein structure capable of incorporating heme. A particularly preferred solubilization buffer according to the invention is buffer Tris pH7.5, CaCl₂, NaCl or Tris pH7.5, CaCl₂, oxidized glutathione.

According to a preferred embodiment, the method further comprises the step of adding heme, which is a co-factor of the polypeptide of the invention, so as to obtain a catalytically active polypeptide, i.e. in a form of a holoenzyme. To this end, heme may be added directly in the culture medium of the host cell and/or in a solubilization buffer such as described above.

The iron in the heme is preferably in a ferric state (Fe³⁺) In a particularly preferred embodiment, the solubilization buffer comprises heme and calcium such as CaCl₂, wherein iron in the heme is preferably in a ferric state (Fe³⁺). For example, one may use a buffer comprising Tris pH7.5, CaCl₂, oxidized glutathione and hemin or a buffer comprising Tris pH7.5, CaCl₂, NaCl and hemin.

Examples of a method allowing the obtention of an isolated polypeptide is described in section 1.6 of the Examples below, as well as in Eggenreich et al. (Biotechnology Reports, 2016, 10: 75-83). Such methods can notably allow the production of high yield of the polypeptide, notably in the form of a holoenzyme. As reported in the following Examples, the Inventors obtained about 30 mg of the polypeptide of the invention from 2 liters of the cultured host cell.

The Inventors have additionally developed a composition in which the activity of the myeloperoxidase-catalase of the invention can be retained, and can accordingly increase its shelf-life. Enzymes such as peroxidase-catalases can indeed be particularly unstable and lose activity over time, notably after freeze-drying. The stabilized composition provided herein is thus particularly suited for the storage of the isolated polypeptide of the invention, whether in a solid form or in a form ready-to-use.

It is thus a further aspect of the invention to provide a composition for stabilizing the polypeptide of the invention, said composition comprising, or consisting of, the isolated polypeptide as described herein and a stabilizing agent.

In a preferred embodiment, the stabilizing agent in this composition is Tris buffer, especially when the polypeptide has a myeloperoxidase activity.

The Inventors notably discovered that Tris buffer has a marked stabilizing effect on the polypeptide over time, especially when such composition is stored between about 4° C. to about 37° C., ideally for at least 20-30 days.

In a preferred embodiment, Tris buffer in said composition is at a pH ranging from about pH7 to about pH8, advantageously at a pH of about 7 or about 7.5.

In a preferred embodiment, the stabilizing composition comprises from about 10 mM to about 100 mM of Tris buffer, preferably from about 20 mM to about 80 mM mM of Tris buffer, more preferably about 50 mM Tris buffer.

In a preferred embodiment, the stabilizing composition comprises from about 15 μM to about 150 μM of the isolated polypeptide, preferably from about 30 μM to about 125 μM of the isolated polypeptide, preferably about 40 μM of the isolated polypeptide.

The stabilizing composition may be in a liquid, semi-liquid, or lyophilized form.

The invention further provides a method for stabilizing the isolated polypeptide of the invention, especially when said polypeptide has a myeloperoxidase activity, said method comprising the step of adding a stabilizing agent in an amount sufficient to stabilize the polypeptide described herein to an aqueous solution comprising said polypeptide and optionally freeze-drying the resulting mixture.

Preferred embodiments for the isolated polypeptide and stabilizing agent as described above apply mutatis mutandis to this stabilization method.

In a preferred embodiment, the stabilization method of the invention further comprises the step of storing the stabilizing composition, at a temperature ranging from about 4° C. to about 37° C., ideally for at least 30 days especially when the composition is in a liquid form or for at least several months or years especially when the composition is in a lyophilized form.

Thanks to its capacity to catalyze (pseudo)halides into (pseudo)hypohalous acids which are known to display potent bactericidal and antiviral activities, the isolated polypeptide of the invention can be used as an antimicrobial agent.

It is thus a further aspect of the invention to provide an antimicrobial composition, comprising the isolated polypeptide as described herein, especially when the polypeptide has a myeloperoxidase activity.

The term “antimicrobial” refers herein to the killing or inhibition of the growth of a microorganism, and accordingly encompasses the terms “bactericidal”, “bacteriostatic”, “virucidal”, “virostatic”, “fungicidal”, “fungistatic”, “parasiticidal” and “parasitistatic”.

Examples of microorganisms that can be killed or of which the growth can be inhibited according to the invention are further detailed below.

Additional components such as an oxygen donor and/or (pseudo)halide may be included, as desired. These components may be provided in a single composition, or may be separated into binary compositions for later mixing prior to use, as may be needed for a particular application. The skilled practitioner will indeed understand that one of these components (and/or corresponding substrate if any) may be left out and provided separately (hence, as a binary compositions) from the polypeptide so to preclude premature reaction and exhaustion of the components.

In a preferred embodiment, the antimicrobial composition, whether single or binary, further comprises an oxygen donor, such as hydrogen peroxide or a source of hydrogen peroxide.

In a preferred embodiment, the oxygen donor is hydrogen peroxide.

Alternatively, hydrogen peroxide may be provided by including in the composition an agent capable of producing hydrogen peroxide in vivo, ex vivo or vitro, i.e. a source of hydrogen peroxide. Particularly useful agents for such purpose include, without limitation, peroxide-generating enzymes, such as peroxide-producing oxidases.

Thus, in a preferred embodiment, the oxygen donor is a source of hydrogen peroxide that is a peroxide-producing oxidase, preferably selected from the group consisting of a glucose oxidase, a galactose oxidase, a glycollate oxidase, a lactate oxidase, a L-gulunolactone oxidase, a L-2-hydroxyacid oxidase, an aldehyde oxidase, a xhantine oxidase, a D-asparate oxidase, a L-amino acid oxidase, a D-amino acid oxidase, a monoamine oxidase, a pyridoxaminephosphate oxidase, a diamine oxidase, a sulphite oxidase, and any combinations thereof. More preferably, the peroxide-producing oxidase is a glucose oxidase.

The Inventors indeed noticed that the antimicrobial activity of the polypeptide of the invention can be greatly potentiated when combined with a peroxide-producing oxidase, such as a glucose oxidase.

A particularly preferred glucose oxidase according to the invention is the glucose oxidase isolated from Aspergillus niger, as described for example by Gao et al. (Biosens Bioelectron, 2009; 25(2):356-61) (e.g. CAS number: 9001-37-0).

The skilled person will readily understand that the antimicrobial composition, whether single or binary, will preferably comprise a suitable substrate for the peroxide-producing oxidase such as glucose, dextrose or saccharose in the case of a glucose oxidase, galactose for a galactose oxidase, glycolate for a glycollate oxidase, lactate for a lactate oxidase, L-gulunolactone for a L-gulunolactone oxidase, (S)-2-hydroxy acid for a L-2-hydroxyacid oxidase, an aldehyde such as acetaldehyde or butyaldehyde for an aldehyde oxidase, xanthine for a xhantine oxidase, D-asparate for a D-asparate oxidase, a L-amino acid for a L-amino acid oxidase, a D-amino acid for a D-amino acid oxidase, a tyramine for a monoamine oxidase, pyridoxamine 5′-phosphate or an amine for a pyridoxaminephosphate oxidase, a diamine for a diamine oxidase, sulfite for a sulphite oxidase; any of the foregoing optionally in the presence of molecular oxygen (O₂).

In a preferred embodiment, when the peroxide-producing oxidase is glucose oxidase, the composition further comprises a glucose or source of glucose and optionally molecular oxygen (O₂).

In a preferred embodiment, the ratio between the isolated polypeptide and the peroxide-producing oxidase in the antimicrobial composition, whether single or binary, is such that the polypeptide is present in excess concentration compared to the peroxide-producing oxidase, especially when the peroxide-producing oxidase is a glucose oxidase.

Still, in a preferred embodiment, the concentration ratio between the polypeptide of the invention and the peroxide-producing oxidase in said composition, whether single or binary, is of at least about 10:1 or about 11:1, more preferably of at least about 12:1 or about 13:1, even more preferably of at least about 14:1 or about 15:1, especially when the peroxide-producing oxidase is a glucose oxidase. The Inventors notably discovered that these particular ratios allowed the achievement of 100% antimicrobial activity.

As explained above, the presence of at least one (pseudo)halide may also be needed in the antimicrobial composition.

In a preferred embodiment, the antimicrobial composition, whether single or binary, further comprises one or more halides or pseudohalides preferably selected from the group consisting of iodide, thiocyanate, bromide, chloride, and any combinations thereof. A particularly preferred (pseudo)halide of the antimicrobial composition according to the invention is thiocyanate, especially when the peroxide-producing oxidase is a glucose oxidase such as the glucose oxidase isolated from Aspergillus niger.

When the (pseudo)halide of said composition is thiocyanate, preferred pH and temperatures are as defined above, more preferably are a pH ranging from about 5 to about 7.5, in particular a pH of about 7.5 (such as 7.4), and/or a temperature of about 35° C. (such as 37° C.), respectively. A particularly preferred concentration of thiocyanate in the antimicrobial composition is of at least about 25 mM.

A particularly preferred concentration of peroxide-producing oxidase in the antimicrobial composition is of at least about 10 nM, especially when the halide is thiocyanate and/or when the peroxide-producing oxidase is a glucose oxidase such as the glucose oxidase isolated from Aspergillus niger.

A particularly preferred concentration of the polypeptide of the invention in the antimicrobial composition is of at least 300 nM, especially when the halide is thiocyanate and/or when the peroxide-producing oxidase is a glucose oxidase such as the glucose oxidase isolated from Aspergillus niger.

For the purpose of the invention, the antimicrobial composition may be in a form suitable for in vitro, ex vivo or in vivo use, depending on the localization of the microorganism to be targeted. The form of the composition will preferably be selected so as to obtain direct contact with the microorganism of interest. For example, should the antimicrobial composition be intended for administration to a subject, the composition may be in a form suitable for oral, nasal, topical, transdermal or parenteral administration, depending on the localization of the microorganism to be targeted.

The antimicrobial composition of the invention may additionally comprise a pharmaceutically acceptable excipient.

As used herein, the term a “pharmaceutically acceptable excipient” means an inactive or inert, and therefore nontoxic, compound of pharmaceutical grade but devoid of pharmacological action itself. Such excipient can be used to improve properties of a composition, such as shelf-life, retention time at the application site, consumer acceptance, etc. It includes, without limitation, surfactants (cationic, anionic, or neutral); surface stabilizers; other enhancers, such as preservatives, wetting or emulsifying agents; solvents; buffers; salt solutions; dispersion medium; isotonic and absorption delaying agents, and the like; that are physiologically compatible.

The skilled person may also wish to combine the antimicrobial composition of the invention with one or more therapeutic agents, either within the composition (single composition), or separately (binary composition). Such therapeutic agents include, for example, anti-bacterial agents, anti-viral agents, anti-fungicides, anti-parasitic agents, and any combinations thereof. For illustration purpose, examples of therapeutic agents suitable for the present invention include, without limitation, penicillins, cephalosporins, carbacephems, cephamycins, carbapenems, monobactams, aminoglycosides, glycopeptides, quinolones, tetracyclines, macrolides, fluoroquinolones, silver, copper, chlorhexidine, polyhexanide, biguanides, chitosan, and/or acetic acid.

The polypeptide, nucleic acid or composition of the invention can be used in a broad range of industrial, pharmaceutical, medical, cosmetics and ecological applications, as well as in the food industry. It can notably be used for any purpose for which prior art myeloperoxidases or catalases have been reported.

For example, as a myeloperoxidase, the isolated polypeptide, nucleic acid or composition of the invention can be useful for obtaining halogenated organic compounds of interest.

Thus, it is a further aspect of the invention to provide an in vitro use of the isolated polypeptide, nucleic acid or composition as described herein, for halogenating a non-halogenated organic compound, especially when the polypeptide has a myeloperoxidase activity.

In other words, the present invention relates to an in vitro method for halogenating a non-halogenated organic compound, said method comprising the step of contacting in vitro the isolated polypeptide, nucleic acid or composition as described herein with a non-halogenated organic compound, especially when the polypeptide has a myeloperoxidase activity.

As used herein, the terms “organic compounds” refers to gaseous, liquid, or solid chemical compounds whose molecules contain carbon.

An example of halogenation of non-halogenated organic compounds (RH) is as follows, using the isolated polypeptide of the invention:

X⁻+H₂O₂+RH+H⁺→RX+2H₂O

wherein RI represents a halogenated organic compound.

Particularly preferred halogenated organic compounds of interest include, without limitation, active organic compounds and chemical intermediates used during organic chemical synthesis, such as desinfectants, nutrients, pesticides, drugs, antibiotics, advantageously plant antibiotics, antioxydants, adhesives, and radiocontrast agents.

For illustration purpose, when the halide is iodide, iodinated compounds of interest can include, without limitation, phenolic compounds (e.g. mono-, di-, tri-, tetra-iodophloroglucinol, dibromoiodophenol and polymers thereof, as well as iodinated phlorotannins such as iodinated fuhalols, phlorethols, fucols, fucophlorethols, eckols and carmalols), volatile hydrocarbon compounds (e.g. iodoform, iodomethane, diiodomethane, bromoiodomethane, iodoethane, iodopropane, iodobutane, etc), terpenes, amino-acids derivatives (e.g. mono-and diiodotyrosine, which are thyroxine precursors) and fatty acids derivatives (e.g. eiseniaiodides). Iodomethane, diiodomethane, iodoform can be used as desinfectants or pesticides. Iodomethane, also known as methyl iodide, can additionally be used as a chemical intermediate during organic chemical synthesis, notably for methylating other compounds such as phenols, carboxylic acids, ammonia and derived amines, and for the industrial-scale production of acetic acid and acetic anhydride.

As another example, when the halide is iodide, radiocontrast agents obtainable by the invention can include, without limitation, 1,3,5-triiodobenzene and derivatives thereof, such as the ionic agents diatrizoate, metrizoate and ioxaglate, and the non-ionic agents ioversol, iopamidol, iohexol, ioxilan, iopromide and iodixanol. Such agents can be used for X-Ray imagery, such as fluoroscopy.

As a myeloperoxidase, the isolated polypeptide, nucleic acid or composition of the invention can also be useful for inhibiting the growth of a wide range of microorganisms, especially those that are pathological, such as those resistant to conventional therapies, in in vitro, ex vivo or in vivo applications.

Accordingly, it is thus a further aspect of the invention to provide an in vitro or ex vivo use of the isolated polypeptide or nucleic acid or composition as described herein, for killing or inhibiting the growth of microorganisms, especially when the polypeptide has a myeloperoxidase activity.

In other words, the present invention relates to an in vitro or ex vivo method for killing or inhibiting the growth of microorganisms, said method comprising the step of contacting in vitro or ex vivo the isolated polypeptide, nucleic acid or composition as described herein with a material or surface contaminated or at risk of being contaminated by microorganisms, especially when the polypeptide has a myeloperoxidase activity.

Such application can indeed be particularly suited to treat materials or surfaces that are contaminated or susceptible to be contaminated with microorganisms, so as to disinfect them, for example prior or after their use. The material or surface may be a surface of any device, laboratory material, surgery material, etc. such as a medical device, contact lenses and the like, especially those are intended to be used in contact with a subject (e.g. sutures, bandages, gauze, staples, zippers, etc.). Materials or surfaces treated or coated with the isolated polypeptide, nucleic acid or composition, as described herein, are also encompassed herein.

By “microorganisms”, it is meant herein bacteria, and also viruses, fungi, and parasites. For illustration purpose, examples of bacteria that can be efficiently inhibited by the present aspect of the invention include, without limitation, a wide range of Gram-negative or Gram-positive, such as Escherichia sp. (such as E. coli), Enterococcus sp., Staphylococcus sp., Streptococcus sp., Citrobacter sp., Enterobacter sp., Klebsiella sp., Proteus sp., Acintobacter sp., Pseudomonas sp., Aeromonas sp., and Pasteurella sp., to name a few, but also Bacillus sp., Clostridium sp. and the like. Examples of fungi that can be efficiently inhibited by the present aspect of the invention include, without limitation, Aspergillus sp., Fusarium sp., Trichophyton sp., and the like. A particularly preferred microorganism according to the invention is a Escherichia sp., such as E. coli.

In addition, since the myeloperoxidase of the invention operates by an entire different mechanism of action than those involved in traditional therapies such as antibiotics, in some instances, the present aspect of the invention may be useful to eliminate drug-resistant, multi-drug resistant, or antibiotics-resistant microorganisms. For illustration purpose, examples of drug-resistant microorganisms include, without limitation, the pathogenic bacteria MRSA (methicillin-resistant Staphylococcus aureus), VRSA (Vancomycin-resistant Staphylococcus aureus), VRE (Vancomycin-ResistantEnterococcus), Penicillin-ResistantEnterococcus, PRSP (Penicillin-resistant Streptococcus pneumoniae), the isoniazid/rifampin-resistant Mycobacterium tuberculosis, and other antibiotic-resistant strains of E. coli, Salmonella, Campylobacter, and Streptococci.

In another aspect, the present invention relates to an isolated polypeptide or nucleic acid or composition as described herein, for use as a medicament, preferably for the treatment of a microbial infection, especially when said polypeptide has a myeloperoxidase activity.

In particular, the present invention is directed to the use of the isolated polypeptide or nucleic acid or composition as described herein, for the preparation of a medicament, preferably for the treatment of a microbial infection, especially when said polypeptide has a myeloperoxidase activity.

The present invention also provides a method for treating a microbial infection in a subject in need thereof, said method comprising the administration of a therapeutically effective amount of an isolated polypeptide or nucleic acid or composition as described herein, to said subject, especially when said polypeptide has a myeloperoxidase activity.

The present invention further relates to (i) an isolated polypeptide or nucleic acid of the invention and (ii) an oxygen donor such as hydrogen peroxide or a source of hydrogen peroxide as described above, as a combined preparation for simultaneous, separate or sequential use as a medicament, preferably for the treatment of a microbial infection, especially when said polypeptide has a myeloperoxidase activity.

Preferred embodiments for the oxygen donor, in particular for the source of hydrogen peroxide, are as described above.

Generally speaking, the term “treatment” or “treating” means obtaining a desired physiological or pharmacological effect depending on the degree of severity of the symptom or disorder of interest, or risks thereof, i.e. herein, depending on the degree of severity of the microbial infection, or risks of developing such symptom or disorder. The effect may be prophylactic in terms of a partial or complete prevention of the symptom or disorder and/or may be therapeutic in terms of a partial or complete cure of the symptom or disorder. The term prophylactic” characterizes the capacity to avoid, or minimize the onset or development of a symptom or disorder before its onset (for example, after exposure the microorganism, but before the onset of associated symptom). The term “therapeutic” refers to the capacity to inhibit the symptom or disorder (i.e. arresting the development thereof), and/or to relieve said symptom or disorder (i.e. regression leading to an improvement). In the context of the invention, a prophylactic effect is generally said to be achieved when e.g. an asymptomatic subject exposed to a microorganism remains asymptomatic or quasi-asymptomatic after treatment according the invention (for example, no development of an infection), while a therapeutic effect is typically said to be achieved when e.g. a symptomatic subject infected with a microorganism recovers after treatment according to the invention (for example, partial or complete relief of the infection).

Due to its wide spectrum of activity, the isolated polypeptide acting as a myeloperoxidase or nucleic acid or composition of the invention may be advantageously used in the treatment of polymicrobial infections. Polymicrobial diseases involve multiple infectious agents and can include complex, complicated, mixed, dual, secondary, synergistic, concurrent, polymicrobial, or coinfections. Polymicrobial diseases include, for example, infections associated with abscesses, AIDS-related opportunistic infections, conjunctivitis, gastroenteritis, hepatitis, multiple sclerosis, otitis media, periodontal diseases, respiratory diseases, and genital infections.

As a myeloperoxidase, the isolated polypeptide, nucleic acid or composition of the invention may also be advantageously used in the treatment of microbial infections that are resistant to traditional therapies. Examples of such infections include those linked to microorganisms that are drug-resistant as described above.

As indicated above, the treatment according to the invention can be achieved by administering a therapeutically effective amount of an isolated polypeptide or nucleic acid or composition as described herein, to a subject in need thereof, using any suitable scheme of administration. For instance, said administration can be performed orally, nasally, topically, transdermally, parenterally, or any combinations thereof, depending on the type of infection affecting the subject. The route of administration will preferably be designed to obtain direct contact of the isolated polypeptide or nucleic acid or composition with the infecting microorganism. The dose and/or scheme of administration can be easily determined and adapted by the skilled practitioner, in accordance with the age, weight and/or severity of the infection from which the subject suffers.

It shall be further understood that the “subject” to be treated according to the invention is preferably a human or animal, more preferably a human.

As a catalase, the isolated polypeptide, nucleic acid or composition of the invention can find many applications in industry, which may require to destroy or detect hydrogen peroxide, or to provide oxygen.

In processes where hydrogen peroxide is used, the polypeptide of the invention can be used as a catalase to destroy residual hydrogen peroxide. For example, in the textile or paper industry, it can be particularly desired to remove hydrogen peroxide after the bleaching step so to avoid bleaching the dyes. As a further example, in the food industry, peroxide removal from pasteurized milk and dairy effluent or beverages before packaging can be particularly useful to avoid any food adulteration or food oxidation.

Thus, it is a further aspect of the invention to provide an in vitro or ex vivo use of the isolated polypeptide, nucleic acid or composition as described herein, for converting hydrogen peroxide into oxygen and water, especially when the polypeptide has a catalase activity.

In other words, the present invention relates to an in vitro or ex vivo method for converting hydrogen peroxide into oxygen and water, said method comprising the step of contacting in vitro or ex vivo the isolated polypeptide, nucleic acid or composition as described herein with a sample containing hydrogen peroxide or a source of hydrogen peroxide, especially when the polypeptide has a catalase activity.

The present invention will be better understood in the light of the following detailed experiments. Nevertheless, the skilled artisan will appreciate that the present examples are not limitative and that various modifications, substitutions, omissions, and changes may be made without departing from the scope of the invention.

### Examples

The Inventors identified a peroxidase from Rhodopirellula baltica, named herein RbMPO. The present study demonstrates that this enzyme is a bacterial homolog of mammalian peroxidases. Indeed, a comparison to human MPO primary and tertiary structure showed that essential amino acids corresponding to the distal and proximal heme cavity or binding site for Ca² were conserved. The overlaying of the Protein Data Bank (PDB) model of hMPO and the structure prediction obtained by Phyre2 for RbMPO showed essential amino acids that are conserved. RbMPO was cloned, produced in E. coli, purified from inclusion bodies and its heme prosthetic group was reconstituted. The biochemical, enzymatical and functional studies of this enzyme demonstrated the surprising broad range of activities displayed by this enzyme, from classic peroxidase activity, production of hypohalides and pseudohypohalides, to a catalase activity. A particular composition allowing the long-term stabilization and conservation of the enzyme was further identified. At last, the microbicidal activity of wt RbMPO was demonstrated against the ATCC 25922 E. coli strain.

### 1. Materials and Methods

1.1. Materials Oligonucleotides were synthesized by Sigma. Plasmid sequencing was performed by Genewiz (Leipzig, Germany). Substrates used to assess the enzyme activity were NaSCN (>99,99%, Sigma), NaBr (>99,99%, Sigma), and NaCl (>99,5%, Sigma BioXtra). Lysogenic Broth (LB) was sourced from MP Biomedicals™ and Tryptic Soy Broth (TSB) from Becton Dickinson. 96-wells plate were from Greiner bio-one. Triton X⁻100, Aminophenylfluorescein (APF) and absolute ethanol were purchased from Sigma. HAZ-TABS 4,5g chlorine (Guest Medical) was used diluted in water as disinfectant. Catalase from bovine liver was purchased from Sigma and the strain E. coli 25922 was sourced from ATCC. Isopropyl b-D-1-thiogalactopyranosil (IPTG) was from Euromedex. ABTS was from Roche.

### 1.2. Cloning of the Wild-Type (Wt) RbMPO

The ORF set forth in SEQ ID NO: 2, encoding the polypeptide SEQ ID NO: 1, was amplified by PCR using the genomic DNA from Rhodopirellula baltica WH47. Genomic DNA was purified with DNeasy® Blood & Tissue from biomass kindly provided by Dr Jens Harder. Genomic DNA was used as a template for the PCR using the primers of SEQ ID NO: 3 and 4. The XhoI and NdeI restriction sites present in these primers were used to clone the gene set forth in SEQ ID NO: 2 into a plasmid derived from pET21a, yielding pET21aRbMPO-6His (His tag in C-Ter) for overexpression under control T7 promoter. The cloned ORF was subsequently sequenced to confirm the absence of unwanted mutations during PCR amplification.

TABLE 4
Primers for cloning of wt RbMPO (F: forward; R: Reverse)

| Primers | Nucleotide sequence (from 5′ to 3′) | SEQ ID NO: |
|---|---|---|
| F (cloning) | GCCAACGTTGTTGCACATCATATGTTGTTCTGGTCG | SEQ ID NO: 3 |
| R (cloning) | GCTTCTACGGTGCCGAACTCGAGAGCGAGGACG | SEQ ID NO: 4 |
| F (wt no tag) | TTTATCGTCCTCGCTTGAGAGCACCACCACCAC | SEQ ID NO: 5 |

1.3. Protein Sequence Alignment of Wt RbMPO with Known Mammalian Peroxidases

A sequence alignment was performed between 5 sequences with Bioedit software 7.2.5 version: the sequence of human myeloperoxidase, human lactoperoxidase, human thyroid peroxidase, human eosinophil peroxidase and RbMPO (SEQ ID NO: 1).

1.4. Modelisation of 3D Structure of the Wt RbMPO, and Superposition with the Structure of Known Mammalian Peroxidases

The 3D structure of wt RbMPO was predicted using the Phyre2 software, using Protein Data Bank (PDB) file of bovine LPO. The predicted structure of wt RbMPO was then superposed with the 3D structure of hMPO as a model on PyMol software 1.7.4 version.

### 1.5. Cloning of Mutated RbMPOs

7 mutated versions of the gene set forth in SEQ ID NO: 2 were generated by site-directed mutagenesis (Quick-Change site-directed mutagenesis kit, Agilent) with the respective primers SEQ ID NO: 5 to 11. Mutations were introduced into amino acids predicted to be key in the active site, based on the results of protein sequence alignment and of overlaying of 3D structure with other mammalian peroxidases.

TABLE 5
Primers for cloning mutated RbMPOs (F: forward; R: Reverse)

| Primers | Nucleotide sequence (from 5′ to 3′) | SEQ ID NO: |
|---|---|---|
| R (cloning) | GCTTCTACGGTGCCGAACTCGAGAGCGAGGACG | SEQ ID NO: 4 |
| F (N317M) | GTTCGTGCCAGCGAGATGGTGGGTTTGACGGCG | SEQ ID NO: 6 |
| F (D202A) | GGGGGCAATTCATCGCGCATGACTTAGGATTGC | SEQ ID NO: 7 |
| F (E316A) | GTTCGTGCCAGCGCGAACGTGGGTTTGACGGCG | SEQ ID NO: 8 |
| F (H407A) | CCGCCGCGTTTCGGTTGGGGGCGAGCACGCTTCGTG | SEQ ID NO: 9 |
| F (H203A) | GGGCAATTCATCGATGCGGACTTAGGATTGACTG | SEQ ID NO: 10 |
| F (Q199A) | GTTTATGTGTGGGGGGCGTTCATCGATCATGAC | SEQ ID NO: 11 |

### 1.6. Production and Purification of Wt and Mutated RbMPOs (as “Apoenzymes” or “Holoenzymes”)

Recombinants plasmids were transformed into E. coli BL21Star (DE3) or C41-pG-KJE8. An overnight culture was used to inoculate 1L of LB supplemented with 100 μg/L ampicillin and 35 μg/L chloramphenicol. The culture was incubated at 37° C. until the exponential growth phase was reached (0.6-0.8 A₆₀₀ₙₘ), then protein expression subsequently induced with 500 μM IPTG followed by 24 h incubation at 22° C. Cells were harvested by centrifugation at 6000×g for 20 min at 4° C., then the pellet was washed with 50 mM Tris pH7.5 buffer, supplemented with antiprotease tablet (Roche), and crushed twice (TS2 0.75 kW Serie Bench Top, Constant systems Ltd., United-Kingdom) at 2200 bar/4° C. For the purification of wt and mutated RbMPOs, pellets were sonicated 10 min and incubated in a urea solution (8 M urea, 20 mM Tris pH7.5, 100 mM NaCl) during 4 h under stirring at 4° C. Another centrifugation step at 21000 xg/4° C./45 min was required and the supernatant underwent successive dialysis steps (6 M>4 M>2 M>0 M of urea) every 12 h. After centrifugation at 21000×g/4° C./45 min and filtration at 0.45 μm, the protein solution was injected onto a Hiload® 16/60 Superdex® 75 μg (Cytiva) previously equilibrated with 50 mM Tris pH7.5 and 5.5 mM CaCl₂)(buffer A). Isocratic elution with the same buffer as equilibration allowed to collect the wt or mutated RbMPO in the dead column volume (size of RbMPO: 78272 Da and column splitting domain: 3000-70000 Da). The enzymatic fraction was diluted in buffer A, and injected in a Resource™ Q 6 mL (Cytiva) pre-equilibrated with 50 mM Tris pH7.5, 5.5 mM CaCl₂ and 1 M NaCl (buffer B). The wt or mutated RbMPOs were eluted with an increasing gradient of buffer B.

This process leads to the production of the proteins in an “apo” form (apoenzyme), i.e. without their coenzyme (herein, heme). The “apo” RbMPOs were eluted with an increasing gradient of buffer B (0-1 M NaCl).

To obtain the proteins in their “holo” form (holoenzyme or haloenzyme), i.e. complexed with their coenzyme (herein, heme), an additional step of reconstitution was performed overnight. “Apo” proteins were incubated with hemin 50 μM, CaCl₂ 5.5 mM, Tris 0.1 M, oxidized glutathione 0.75 mM for 24h on wheel at 4° C.

Purified proteins (whether “apo” or “holo”) were then freeze-dried and stored at 4° C. after ultrafiltration and diafiltration against a 50 mM pH 7.5 TRIS buffer without any trace of chloride. All purification steps were made using an Akta system (GE Healthcare). Enzyme concentration was determined spectrophotometrically, using the theoretical epsilon of RbMPO (wt and mutated): ε=49850 M⁻¹.cm⁻¹ and absorbance at 280 nm.

1.7. Reconstitution of the Active Site of Wt and Mutated RbMPOs (from “Apoenzymes” To “Holoenzymes”)

Tryptophan fluorescence quenching and recovery can be used to monitor the protein confirmation changes from an “apo” to an “holo” form upon addition of its coenzyme (herein heme).

The Trp fluorescence quenching experiment was performed in a spectrofluorometric FP8300 with a temperature-controlled stirred-cell (25° C.). Increasing concentrations of heme were used (0-60 μM) and the fluorescence intensity of apo RbMPOs was measured at 450-300 nm by exciting the sample first at 297 nm, then at 334 nm as a function of time. Sample dilutions were performed in a 50 mM pH7.5 sodium phosphate buffer.

### 1.8. Inhibition of the Wt and Mutated RbMPOs by ABAH (“Holoenzymes”)

The halogenating activity of myeloperoxidases can be determined using the aminophenyl fluorescein (APF) probe. One can indeed detect the oxidation of APF (non-fluorescent) by Cl⁻ into fluorescein, a fluorescent molecule that is detectable at λₑₓ=485 nm and λₑₘ=525 nm. According to such test, the fluorescence measured is proportional to the quantity of fluorescein, which in turn is proportional to the quantity of Cl⁻ produced by the myeloperoxidase. 4-aminobenzoic acid hydrazide (ABAH) is a well-known potent inhibitor of hypochlorous acid production, and can accordingly be used to inhibit chlorination activity.

The activity of the RbMPO was assessed herein in the absence and presence of an increasing range of ABAH 0, 0.5, 0.1, 0.25, 0.5, 0.6, 0.7, 0.8, 0.9, 1, 5, 50, 100, 500 and 1000 μM) with fixed concentrations of NaCl (500 mM), H₂O₂(500 μM), RbMPO enzyme (1 μM) and APF (10 μM). Fluorescence emission was measured at 525 nm λₑₓ=485 nm). Measurements were performed in Coming 384-well clear-bottom (flat-bottom) plates with a reaction volume of 20 μL. Each measurement was performed in triplicate at 37° C., and in 50 mM NaPi buffer pH7.5. 10-minutes kinetic was performed for each value of the ABAH range.

Tryptophan fluorescence quenching and recovery can be used to monitor the protein confirmation change from an “holo” to an “apo” form following ABAH treatment (ABAH can typically disrupts the covalent bond between the heme and the enzyme).

Trp Fluorescence recovery was done by using an ABAH range (0-1 mM) applied on holo RbMPOs at 1 μM per essay (diluted in a 50 mM pH7.5 sodium phosphate buffer). The fluorescence intensity of holo RbMPOs was measured by exciting the sample at 297 nm and first measuring the fluorescence intensity from 450-300 nm first, followed by kinetics at 334 nm. All experiments were performed in a spectrofluorometric FP8300 with a temperature-controlled stirred-cell at 25° C.

### 1.9. Steady-State Kinetic Parameters of Wt and Mutated RbMPOs (“Holoenzymes”)

Initial rate measurements under steady state conditions were performed with a BioLogic SFM400 stopped-flow by using a single-mixing mode and an Xe/Hg lamp and a TC100/10 cell. One syringe was filled with RbMPO and the other one with or without taurine, H₂O₂ and NaBr or NaSCN or NaCl. The appearance of taurine bromamine (Tau-NHBr), taurine chloramine (Tau-NHCl) and hypothiocyanate (OSCN) were measured at 289, 253 and 240 nm respectively at 37° C. in the presence of a 50 mM pH 7.5 sodium phosphate buffer. At least five 120 s shots were monitored for each value of the substrate range with the following sample rates: 20 μs, 20 ms, 20 ms with 2000 points per period. Slopes were calculated from the average of the shorts for each concentration and kₛₛ (s⁻¹) were determined from the slopes using the following formula:

$${k}_{ss} = \frac{slope ( Abs \cdot {s}^{- 1} )}{{ε}_{product} ( {M}^{- 1} \cdot {cm}^{- 1} )} \frac{1}{[ RbMPO ] ( M )} \frac{1}{l ( 1 cm )}$$

The concentrations in Wt and mutated RbMPOs in the test were of 300 nM when NaBr or NaSCN were used as (pseudo)halides, and of 2 μM with NaCl as a halide, ; molar extinction coefficients for each product were ε_{Tau-Br}=415 M⁻¹.cm⁻¹, ε_{Tau-Cl}=429 M⁻¹.cm⁻¹ and ε_{OSCN}=951 M⁻¹.cm⁻¹. The collected data were analysed with Origin software, then saturation curves for each substrate were fitted either by non-linear regression with or without inhibition by excess of substrate (Eq. 1 and 2) with [S]the substrate of interest, or by linear regression (Eq. 3) with a the slope and b the y-intercept.

$${k}_{ss} = \frac{{k}_{cat} * [ S ]}{{K}_{M} + [ S ]} \qquad Eq . 1$$

$${k}_{ss} = \frac{{k}_{cat} * [ S ]}{{K}_{M} + [ S ] * ( 1 + \frac{[ S ]}{{K}_{I}} )} \qquad Eq . 2$$

$${k}_{ss} = {k}_{2} * [ S ] + b \qquad Eq . 3$$

pH- and temperature-dependent studies were carried out for each substrate.

For the temperature study, wt RbMPO was incubated at 4° C. with kinetics measurements performed at increasing temperatures: 10, 20, 30, 37, 40, 50 and 60° C. These temperatures were reached by using a stopped-flow coupled to a cryostat. Substrate concentration was set at 79 μM for H₂O₂, at substrate saturation for NaSCN and NaBr, and at 500 mM for NaCl.

For the pH study, a range of pH was assessed: 4, 5, 6, 7, 8, 9, 10, and RbMPO was solubilized in different buffers in which the ionic strength did not vary (citrate phosphate pH4 (0.1 M citrate, 0.2 M phosphate); 0.163 M sodium acetate pH5; 0.89 M potassium phosphate pH6; 0.0566 M potassium phosphate pH7; 0.0162 M sodium phosphate pH8; 0.0136 M pyrophosphate pH9; 0.0107 M pyrophosphate pH1O). Similar to the temperature experiment, substrate concentration was set at 79 μM for H₂O₂, 100 mM for NaBr, 5 mM for NaSCN and 100 mM for NaCl.

For temperature and pH measurements, RbMPO was set at the same concentration as in the determination of steady state kinetics parameters.

### 1.10. Wt RbMPO (“Holoenzyme”) Kinetics Parameters in Pre-Steady State

In the first moment after an enzyme is mixed with a substrate, no product has been formed and no intermediates exist. The study of the next few milliseconds of the reaction is called pre-steady-state kinetics.

Kinetic parameters under pre-steady-state condition were performed with a Biologic SFM400 stopped-flow at 412 nm, 37° C. and with 50 mM sodium phosphate buffer at pH7.5. The formation of compound I of wt RbMPO was demonstrated by measuring the absorbance decay time at 412 nm in the presence of increasing H₂O₂ concentration under single turnover conditions. An exponential linear fit was applied to each kinetic using the following formula:

$$y = {p}_{1} {e}^{\frac{- x}{{p}_{2}}} + {p}_{3} + {p}_{4} x \qquad Eq . 4$$

where p₁ corresponds to the exponential magnitude, p₂ to the decay time Tau (s) and

$${k}_{obs} = \frac{1}{Tau} ( {s}^{- 1} ) ,$$

p₃ a constant and p₄ the slope.

The return of the enzyme to the native state was shown with increasing concentrations of NaBr, NaSCN, NaCl and NaI in the presence of compound I.

### 1.11. Chlorination Activity of Wt and Mutated RbMPOs (“Holoenzymes”)

The chlorination activity of myeloperoxidases can be determined using the aminophenyl fluorescein (APF) probe. One can indeed detect the oxidation of APF (non-fluorescent) by HOCl into fluorescein, a fluorescent molecule that is detectable at λₑₓ=485 nm and λₑₘ=525 nm. According to such test, the fluorescence measured is proportional to the quantity of fluorescein, which itself is proportional to the quantity of HOCl produced by the myeloperoxidase.

Measurements of chlorination activity were herein performed in Corning 384-well clear-bottom (flat-bottom) plates with a reaction volume of 20 μL, in triplicate at 37° C., and in a 50 mM NaPi buffer at pH7.5. For each enzyme (wt and mutants), two tests were investigated at different NaCl or H₂O₂ concentration ranges: a first test with NaCl (0-1 M),1 μM RbMPOs, 79 μM H₂O₂ and 10 μM APF; a second test with 500 mM NaCl, H₂O₂(0-500 mM), 1 μM RbMPOs and 10 μM APF. A 10 to 30 minutes kinetic was performed for each range value and the fluorescence intensity was compared to that of the standard fluorescein range. Thus, the measured fluorescence is proportional to the amount of fluorescein, which in turn is proportional to the amount of HOCl produced by the RbMPOs.

### 1.12. Catalase Activity of Wt RbMPO (“Holoenzyme”)

Catalase activity was measured using the stopped-flow set-up, which was configured to measure absorbance decay at 240 nm and at 37° C.

An increasing range of H₂O₂ concentration (0, 0.02, 0.05, 0.1, 0.25, 0.5, 0.75, 1, 2, 5, 10 mM) was used in presence of 2 μM of wt RbMPO, at different pH values (4, 5, 6, 7, 7.5, 8, 9 and 10) with the same buffers as for the pre-steady state study described in above section 1.10.

A similar experiment was performed at 300 nM wt RbMPO at different temperatures (10, 20, 30, 37, 40, 50 and 60° C.) at pH 7.5 and at fixed concentration of H₂O₂(500 μM).

### 1.13. Peroxidase Activity of Wt RbMPO (“Holoenzyme”)

ABTS (2,2′-azino-bis(3-ethylbenzothiazoline-6-sulfonic acid) is a chemical compound commonly used as a substrate with hydrogen peroxide to assess the reaction kinetics of peroxidases, and of which the soluble-end product of the reaction can be easily detected (it is green colored, and detectable at 420-490 nm).

Measurements were performed herein in a 384-well plate (Corning® 384 well microplate) at 37° C., in triplicate, at fixed concentration of wt RbMPO (0.5 nM) and H₂O₂(79 μM) in a final volume of 20 μL in 50 mM NaPi buffer pH5.5. The plate reader was configured to run a 10 min kinetics at 430 nm with the following ABTS concentration range: 0, 0.1, 0.2, 0.5, 1, 2, 5, 10, 15, 20, 30 and 40 mM.

### 1.14. Stabilization Conditions for Storage of Wt RbMPO (“Holoenzyme” or “Apoenzyme”)

The stability of wt RbMPO in various buffers was assessed over time under different storage conditions.

In a first experiment, the wt RbMPO was stored for up to 30 days, in a 50 mM Tris buffer pH 7.5 at two temperatures, namely 4° C. and 37° C. The enzyme was concentrated at 123.4 M for the 4° C. storage condition and at 132.2 μM for the 37° C. storage condition. The MPO activity of the enzyme was assessed at different time points by a standard activity test. To do so, the enzyme was diluted and contacted with H₂O₂(79 μM), NaCl (500 mM) and APF (10 μM).

In a further experiment, the wt RbMPO was stored for up to 60 days, in a 50 mM NaPi buffer pH 7.5, also at 4° C. and 37° C. The MPO activity of the enzyme was assessed at different time points by a standard activity test. To do so, the concentrated enzyme was diluted and contacted with H₂O₂, NaCl and APF.

In a further experiment, the wt RbMPO was stored for over 40 minutes in a PBS buffer pH 6.6, at 37° C. The MPO activity of the enzyme was assessed at different time points by a standard activity test. To do so, the concentrated enzyme was diluted and contacted with H₂O₂, NaCl and APF.

### 1.15. Microbicidal Activity of Wt RbMPO (“Holoenzyme”)

The microbicidal activity of RbMPO towards the E. coli strain ATCC 25922 was evaluated by two independent experiments. This E. coli strain is typically recommended for conducting antibiograms/for assessment of antimicrobial activity of a compound of interest.
By measuring bacterial growth kinetics in a microplate

E. coli 25922 were grown on TSB-agar for 16 h at 37° C. One colony is grown in 10 ml of TSB medium under stirring at 190 rpm at 37° C. The optical density (OD) of the bacterial preculture was measured at 620 nm in a spectrophotometer (Ultrospec 10, Biochrom). 25 ml of TSB medium was inoculated to obtain an OD of approximately 0.09. When the OD was between 1 and 2, a dilution in 10 mL of TSB medium was performed to obtain 2×10⁶ CFU/ml. Bactericidal tests were performed in 96-well microplates under a final volume of 100 μl with 10⁶ CFU/ml. The plate cover was treated with 0.05% TritonX−100 and 20% ethanol to prevent condensation and thus avoid misreading of the wells by the instrument. The solution was allowed to act on the lid for 10 minutes, then removed and dried under a class II biological safety cabinet. Each tested condition was performed at least in triplicate and each experiment was repeated at least 3 times. The kinetics was set to 16 h with a measurement at 620 nm every 15 minutes in a microplate reader (Wallac Victor 2,1420 Multilabel counter) thermostated at 37° C. [shaking duration: 10.0s; shaking speed: fast; shaking diameter: 0.5 mm; shaking type: orbital; measurement time: 0.2s; numbers of replicates: 60; time between replicates: 900 s; label: P620]. Enzymes essays were performed: first with GOx_{aspniger} alone (concentration ranging from 10 nM to 30 nM), then wt RbMPO alone (concentration ranging from 6.25 nM to 2000 nM), and finally with the coupled enzymatic system. For all these experiments, substrates such as NaCl, NaBr, NaSCN or NaI and glucose (for GOx_{aspniger} only or the coupled enzymatic system) were used at fixed concentration. 5 mM bleach was used as a positive control and bacteria alone was used as a negative control.
By counting colonies on Petri dishes

10 mL of TSB medium (pH7.4) was inoculated with a freshly obtained colony of E. coli ATCC 25922, and the culture was incubated at 37° C. and 190 rpm overnight. In the morning, 25 mL of TSB medium were inoculated with the previous day's pre-culture to obtain an OD620 nm of 0.07 at TO. When the bacterial culture reached the exponential phase, the bacterial solution was diluted 10-fold in TSB in order to reach 1.10⁶ CFU/mL. This dilution was then contacted with the enzyme solution for 1 hour (in a final volume of 1 mL). Further dilutions were then made (10⁻¹, 10⁻⁴, 10−5, 10⁻⁶). 100 μL of these solutions were spread on a Petri dish containing TSB agar medium and placed at 37° C. overnight. The next day, the surviving colonies were counted and the number of CFU/mL was calculated using the formula below:

$$\frac{CFU}{mL} = \frac{{Nb}_{colony} * 10}{dilution}$$

The experimental conditions were: GOx_{aspniger}: 0 or 25 nM, wt RbMPO: 0 or 300 nM, and NaSCN 25 mM.

### 2. Results

### 2.1. Production, Purification of Wt and Mutated RbMPOs

This first strategy involved purifying the cell-shredded supernatant. 9 mg of total enzyme (2 L) was obtained in the case of RbMPO-6His. Comparing the production of RbMPO-6His in the BL21Star and C41-pG-KJE8 strains, the expression of enzyme was much greater in the pellet of the 1^{st} strain (BL21Star). With this type of production protocol, the enzyme was localised in inclusion bodies: Wt RbMPO was indeed present in bacterial pellet. Incubation for 4 h at 4° C. in 8 M urea was sufficient to resolubilise RbMPOs. A broad blue band around 75 kDa was present in the supernatant after urea treatment. After the first purification step, wt RbMPO was injected on a second column (Resource™ Q 6 mL). Four peaks were eluted: the 1^{st} one did not contain the enzyme, the 2^{nd} and the 3^{rd} contained the wt RbMPO and the last one did not contain any enzyme. This protocol allowed the production of about 30 mg of enzyme for 2 litters of bacterial culture.

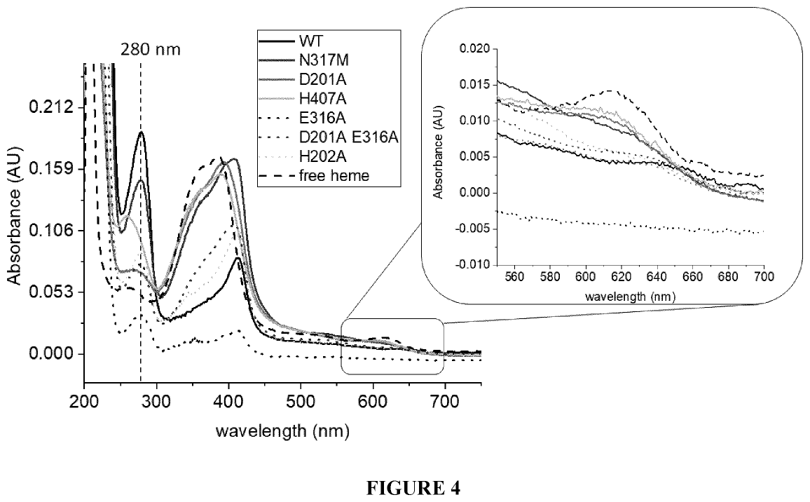

The UV-visible spectrum showed two different peaks of the holoenzymes (FIG. 4). The peak at 280 nm was characteristic of tyrosine and tryptophan amino acids, which are components of RbMPO. Heme reconstitution of the enzyme was evidenced by the presence of a Soret band at 412 nm as shown on the wt RbMPO spectrum (Table 6). Active site mutants, such as (D202A) and (H407A), showed a leftward shift of the peak, at 393 nm and 389 nm respectively; a small peak at 615 nm like free heme was also visible. For these mutants, a change in the binding of the prosthetic group must take place. For the others mutants, namely (D202A, E316A), (E316A), (N317M), (H203A) and (Q199A), a peak close to that of the native form could be observed. The R_{z} ratio (Aₕₑₘₑₚₑₐₖ/A₂₈₀ₙₘ) of wt RbMPO was 0.43.

TABLE 6
Soret band for each form of RbMPO (“holoenzymes”
except free heme)

| Enzymes | Soret band (heme presence) |
|---|---|
| wt RbMPO | 412 nm |
| N317M | 410 nm (and 615 nm) |
| D202A | 393 nm (and 615 nm) |
| H407A | 389 nm (and 615 nm) |
| E316A | 414 nm |
| D202A, E316A | 409 nm |
| H203A | 413 nm |
| Q199A | 412 nm |
| Free heme (wt “apo”) | 385 nm (and 615 nm) |

Investigation of the oligomerisation form of wt RbMPO showed that there was two forms of this enzyme after purification (data not shown). The first elution peak of superose 12 appeared to be in the splitting domain. The 2^{nd} peak appeared to be a monomeric form of wt RbMPO determined by calculation of the partition coefficient and apparent molecular weight (data not shown). These results are consistent with Dynamic Light Scattering (DLS) analysis where most of the RbMPO is in monomeric form with a small amount of aggregates or multimeric forms are present in the sample (data not shown).

### 2.2. Reconstitution of the Active Site of Wild-Type (Wt) and Mutated RbMPOs

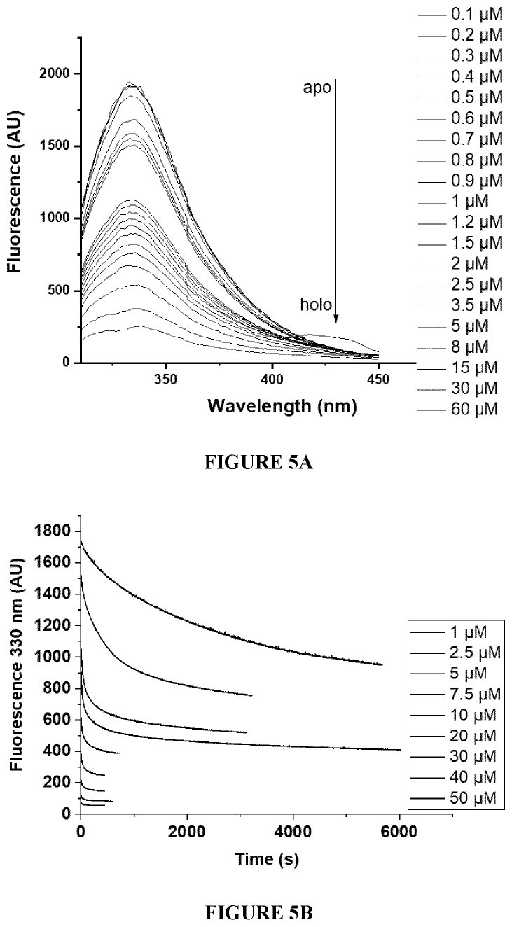

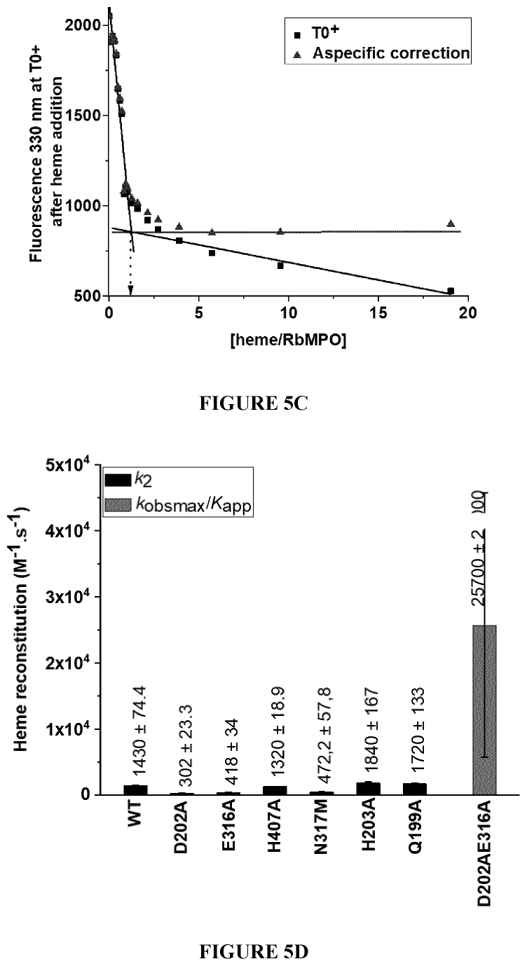

Scanning measurements (450-300 nm) after excitation at 297 nm showed a fluorescence peak at 330 nm (FIG. 5A). An increase in heme concentration resulted in a quenching of the fluorescence intensity at this peak. This means that there was conformational rearrangement in the 3D structure, which is represented by a decrease in tryptophan availability followed by quenching of tryptophan fluorescence emission. The same experiment was performed with active site mutants of the RbMPO active site. Kinetics experiments at 334 nm (or 332 nm for the mutant (D202A)) confirmed heme binding to RbMPOs (FIG. 5B). These experiments allowed the observation of the transition of these enzymes from their apo form to their holo form. Analysis of FIG. 5B with the determination of fluorescence intensity at 334 nm at T0 (right after heme addition) for each heme concentration provided a site titration (FIG. 5C).

The slope of the aspecific correction met at one point. By plotting the line on the x-axis, it could be concluded that one molecule of heme was bound per monomer of RbMPO. The monoexponential analysis in FIG. 5B allowed the calculation of k₂ values by the k_{obs}/[heme]ratio and the representation of the rearrangement following the heme entrance efficiency of wt or mutated RbMPOs (data not shown). The wt RbMPO, as well as the mutants (H407A), (H203A) and (Q199A) had an environment that favored efficient reconstitution with heme, with a k₂ of 1430±74.4 M⁻¹.s⁻¹, 1320±18.9 M⁻¹.s⁻¹, 1840±167 M⁻¹.s⁻¹ and 1720±133 M⁻¹.s⁻¹ respectively (FIG. 5D and Table 7). The (D202A) mutant surely had a different active site that prevents heme entry with a k₂=302+23.4 M⁻¹.s⁻¹. A similar result was observed for the mutants (N317M) and (E316A) with a k₂ of 470.2+57.8 M⁻¹.s⁻¹ and 418+34 M⁻¹.s⁻¹. The double mutant (D202A, E316A) showed a completely different curve profile with saturation of the active site of the enzyme by heme (data not shown): Here, the k_{obsmax}/K_{D} ratio was determined to be 2.57 10⁴±2.00 10⁴ M⁻¹.s⁻¹. By contrast, for all other mutants as well as for the wt RbMPO, the increase in k_{obs} was linear.

TABLE 7
Catalytic constants of wt and mutated
RbMPOs (heme reconstitution)

| Enzymes | k₂ (M−1 · s−1) |
|---|---|
| wt RbMPO | 1430 ± 74.4 |
| D202A | 302 ± 23.3 |
| E316A | 418 ± 34 |
| H407A | 1320 ± 18.9 |
| N317M | 472.2 ± 57.8 |
| H203A | 1840 ± 167 |
| Q199A | 1720 ± 133 |

| | K_{D} (μM) | K_{obsmax} (s⁻¹) | K_{obsmax}/K_{D} (M⁻¹ · s⁻¹) |
|---|---|---|---|
| D202A, E316A | 10.87 ± 6.14 | 0.28 ± 0.06 | 2.57 10⁴ ± 2.00 10⁴ |

### 2.3. Inhibition of the Wt and Mutated RbMPOs by ABAH

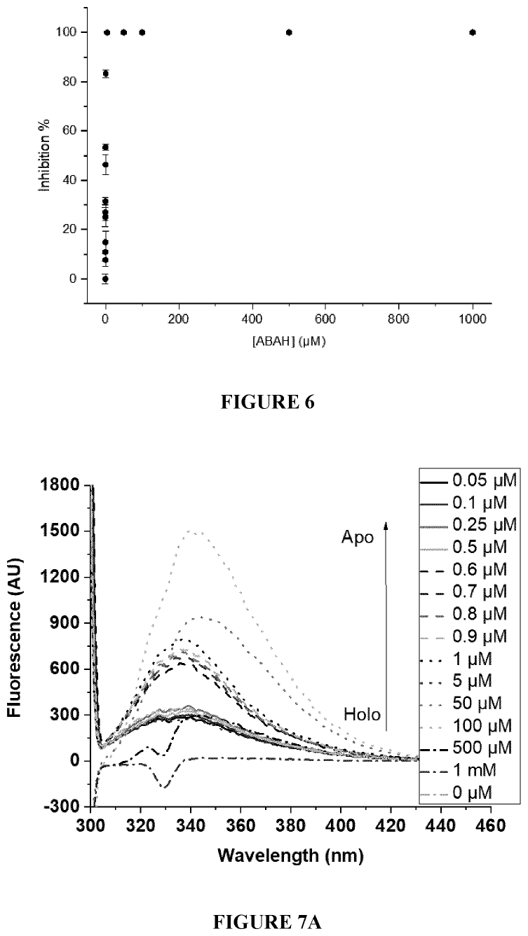

ABAH is known to be irreversible inhibitor of native hMPO. To determine whether ABAH also inhibited wt RbMPO, the chlorination activity of this enzyme was measured in the presence of an increasing ABAH concentration range. The appearance of fluorescein was measured at 525 nm and indicated the formation of the enzyme reaction product, namely HOC. FIG. 6 shows the inhibition percentage of chlorination activity of wt RbMPO. The activity of the enzyme decreased, as the concentration of ABAH increased. At 5 μM of ABAH, 100% of wt RbMPO activity was inhibited. ABAH can thus be considered as an inhibitor of the RbMPO bacterial enzyme.

The experiment highlights the transition of the RbMPOs from their holo form to their apo form. The action of ABAH was indeed to remove the heme from the active site of the enzymes in an irreversible manner, going as far as their degradation.

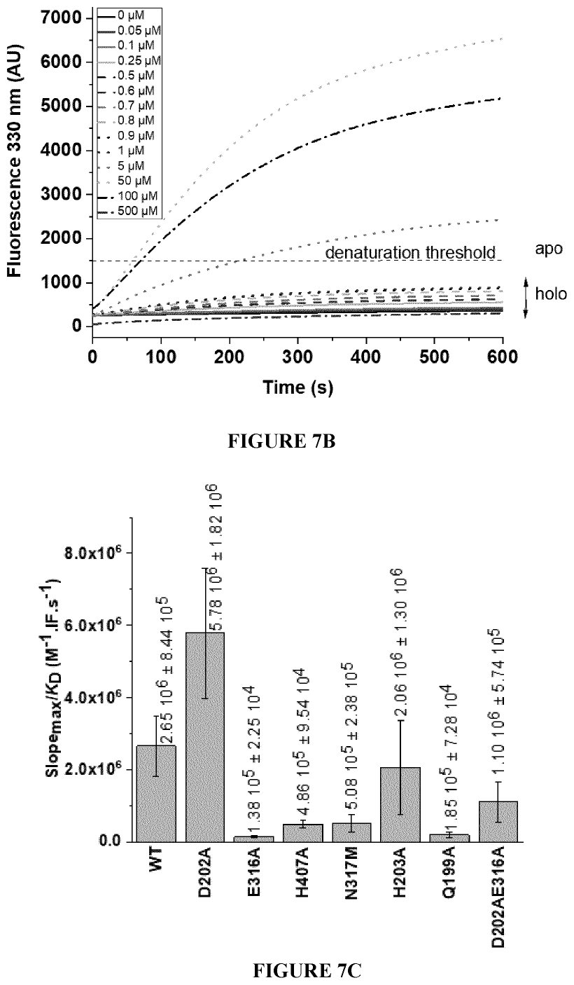

Indeed, FIG. 7A shows that the increase in ABAH concentration from 0 to 1 μM leaded to an increase in fluorescence at a peak of 330 nm for wt RbMPO. At 50 μM and 100 μM of the inhibitor, the peak shifted to the right at 342 nm as the fluorescence intensity continued to increase. The two highest concentrations of ABAH (500 μM and 1 mM) showed a collapse in fluorescence intensity. It was thus hypothesised that the wt enzyme was degraded at these ABAH concentrations. Kinetics at 337 nm (FIG. 7B) confirmed the previous result. Increasing the concentration of the inhibitor increased fluorescence intensity as a function of time up to 50 μM ABAH; fluorescence then decreased at higher concentration of the inhibitor. FIG. 7C shows curves that all have a Michaélis-Menten profile with different k_{obsmax}/K_{D} ratio. The mutant (D202A) had the highest ratio with 5.78 10⁶±1.82 10⁶ M¹.s⁻¹ (Table 8). It was followed by the wt RbMPO (2.65 10⁶+8.44 10⁵ M⁻¹.s⁻¹) then by the mutant (H203A) (2.06 10⁶±1.30 10⁶ M⁻¹.s⁻¹). Interestingly, there was a factor of 50 between the K_{D} of these two forms of RbMPO and a factor of 63 between the k_{obsmax}, whereas the k_{obsmax}/K_{D} ratios were almost identical. The double mutant (D202A, E316A) had a 2.4-fold lower ratio than the wild-type (1.10 10⁶±5.74 10⁵ M⁻¹.s⁻¹). Finally, the mutants N317M (5.08 10⁵±2.38 10⁵ M⁻¹.s⁻¹), H407A (4.86 10⁵±9.54 10⁴ M⁻¹.s⁻¹), Q199A (1.85 10⁵±7.28 10⁴ M⁻¹.s⁻¹) and E316A (1.38 10⁵±2.25 10⁴ M⁻¹.s⁻¹) had the lowest k_{obsmax}/K_{D} ratios which may indicate a slightly slower release of the heme from the active site compared to the wt RbMPO.

TABLE 8
Catalytic constant of wt and mutated RbMPOs (ABAH)

| | K_{D} | K_{obsmax} | K_{obsmax}/K_{D} |
|---|---|---|---|
| Enzymes | (μM) | (s⁻¹) | (M⁻¹ · s⁻¹) |
| wt RbMPO | 7.41 ± 1.88 | 19.64 ± 1.27 | 2.65 10⁶ ± 8.44 10⁵ |
| D202A | 5.72 ± 1.38 | 34.19 ± 2.17 | 5.78 10⁶ ± 1.82 10⁶ |
| E316A | 1.38 ± 0.17 | 0.19 ± 7.70 10⁻³ | 1.38 10⁵ ± 2.25 10⁴ |
| H407A | 7.99 ± 1.24 | 3.88 ± 0.16 | 4.86 10⁵ ± 9.54 10⁴ |
| N317M | 2.93 ± 1.06 | 1.49 ± 0.16 | 5.08 10⁵ ± 2.38 10⁵ |
| D202A, | 8.01 ± 2.62 | 11.25 ± 0.92 | 1.10 10⁶ ± 5.74 10⁵ |
| E316A | | | |
| H203A | 0.15 ± 0.08 | 0.31 ± 0.03 | 2.06 10⁶ ± 1.30 10⁶ |
| Q199A | 1.86 ± 0.56 | 0.35 ± 0.03 | 1.85 10⁵ ± 7.28 10⁴ |

### 2.4. Steady-State Kinetic Parameters of Wild-Type (Wt) and Mutated RbMPOs

The stopped-flow was used to define the steady-state kinetic parameters of RbMPOs. For each (pseudo)-halogenated substrate, a concentration range was used at fixed concentrations of enzyme and H₂O₂. The appearance of the enzymatic product was measured at different wavelengths depending on the substrate: at 240 nm with direct measurement for OSCN formation, while at 253 nm and 289 nm, Tau-NHCl or Tau-NHBr were measured respectively. From the averages of the curves obtained for each shot, the slopes in Abs.s⁻¹ were calculated and then converted into kₛₛ by the following relation:

$${k}_{ss} = \frac{slope}{{ε}_{- OX}} \frac{1}{[ RbMPO ]}$$

The kₛₛ curves as a function of halide Br⁻ and pseudo-halide SCN⁻ concentrations showed Michaëlis-Menten type profiles with saturation of the enzyme by the substrate, a linear increase of kₛₛ depending on increasing substrate concentration, or inhibition by the substrate depending of the enzyme form (data not shown).

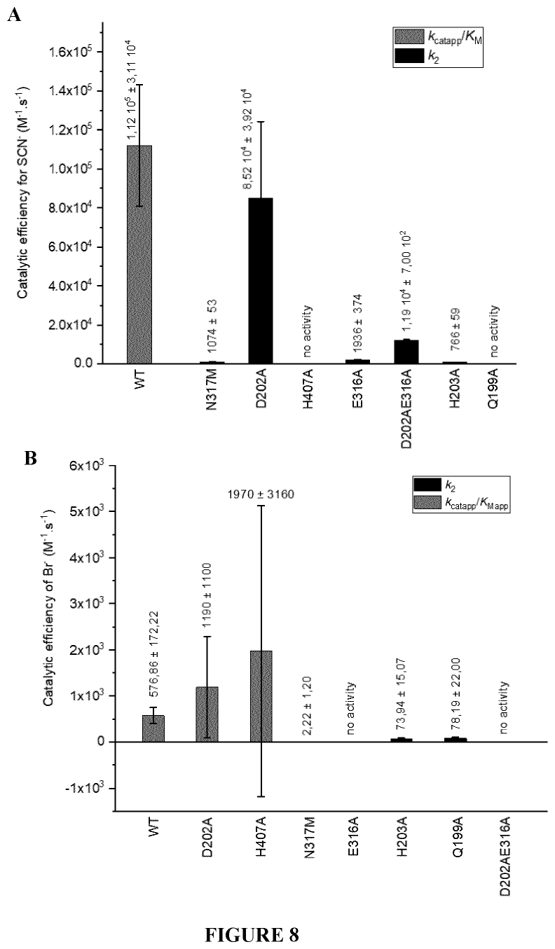

In the case of the measurement of the appearance of ⁻OSCN, for the mutated enzymes (N317M), (E316A), (D202A, E316A) and (H203A), there was a linear increase in the kₛₛ values with the application of a linear regression line. It was then possible to determine their respective k₂: 1074±53 M⁻¹.s⁻¹, 1936+374 M⁻¹.s⁻¹, 1.19 10⁴+7.00 10² M⁻¹.s⁻¹ and 66+59 M⁻¹.s⁻¹, respectively (Table 9 and FIG. 8A). For the wt RbMPO and the mutant (D202A), the curves kₛₛ versus NaSCN concentration showed a Michaëlis-Menten type profile with a ratio k_{catapp}/K_{Mapp} 1.31 times higher for the wt form (1.12 10⁵±3.11 10⁴ M⁻¹.s⁻¹) than for the mutant (D202A)(8.52 10⁴+3.92 10⁴ M⁻¹.s⁻¹). The truncated form of RbMPO (E316A mutant), was active with SCN⁻ with a ratio 1936+374 M⁻¹.s⁻¹. The mutants (Q199A) and H407A were not active in presence of SCN.

TABLE 9
Kinetic parameters of wt and mutated RbMPOs for the
NaSCN range at 300 nM of enzyme and 79 μM of H₂O₂

| | Steady state (NaSCN range) | | | |
|---|---|---|---|---|
| | K_{Mapp} | k_{catapp} | k_{catapp}/K_{MappNaSCN} | k₂ |
| Enzymes | (mM) | (s⁻¹) | (M⁻¹ · s⁻¹) | (M⁻¹ · s⁻¹) |
| wt RbMPO | 0.51 ± 0.08 | 56.90 ± 6.92 | 1.12 10⁵ ± 3.11 10⁴ | n.a |
| N317M | n.a | n.a | n.a | 1074 ± 53 |
| D202A | 0.98 ± 0.31 | 83.54 ± 12.04 | 8.52 10⁴ ± 3.92 10⁴ | |
| H407A | scatter plot | | | |
| E316A | n.a | n.a | n.a | 1936 ± 374 |
| D202A, | n.a | n.a | n.a | 1.19 10⁴ ± 7 10² |
| E316A | | | | |
| H203A | n.a | n.a | n.a | 766 ± 59 |

| | |
|---|---|
| Q199A | No activity |

(n.a.: not available).

Measurement of the onset of Tau-NHBr was not possible for the mutants (E316A) and (D202A, E316A) because of their lack of RbMPO activity with this substrate. For the mutants (D202A) and (H407A), there was an inhibition of the enzyme by Br⁻ with inhibition constants of 69.77±38.05 mM and 47.22±42.87 mM, respectively (Table 10). k_{catapp} K_{Mapp} (M⁻¹.s⁻¹) ratios were very similar for these two mutants (D202A: 1.19 10³±1.10 10³ M⁻¹.s⁻¹ and H407A: 1.97 10³±3.16 10³ M⁻¹. s⁻¹), and were much higher than wt RbMPO (3-fold) and the mutant (N317M) (711-fold) (FIG. 8B). The mutants (H203A) and (Q199A) exhibited more and less the same k₂ with 73.94±15.07 M⁻¹.s⁻¹ and 78.19+22.00 M⁻¹.s⁻¹, respectively; these were 7 times less efficient for Br⁻ than wt RbMPO.

TABLE 10
Kinetic parameters of wt and mutated RbMPOs for the
NaBr range at 300 nM of enzyme and 79 μM of H₂O₂

| | Steady state (NaBr range) | | | | |
|---|---|---|---|---|---|
| | | | k_{catapp}/ | | |
| | | | K_{MappNaSCN} | kₛₛ/[SCN⁻] | |
| Enzymes | K_{Mapp} (mM) | k_{catapp} (s⁻¹) | (M⁻¹ · s⁻¹) | (M⁻¹ · s⁻¹) | Kᵢ (mM) |
| Wt RbMPO | 133.32 ± 32.79 | 76.33 ± 5.12 | 576.86 ± 172.22 | n.a | n.a |
| N317M | n.a | n.a | n.a | 2.22 ± 1.20 | n.a |
| D202A | 48.84 ± 25.20 | 58.29 ± 24.08 | 1.19 10³ ± 1.10 10³ | n.a | 69.77 ± 38.05 |
| H407A | 44.40 ± 41.24 | 87.69 ± 59.05 | 1.97 10³ ± 3.16 10³ | n.a | 47.22 ± 42.87 |
| E316A | No activity | | | | |
| D202A, | No activity | | | | |
| E316A | | | | | |
| H203A | n.a | n.a | n.a | 73.94 ± 15.07 | n.a |
| Q199A | n.a | n.a | n.a | 78.19 ± 22.00 | n.a |

(n.a.: not available).

For practical reasons, only wt RbMPO was studied by stopped-flow with a choride range to determine the steady state kinetic parameters. The kₛₛ values increased linearly with Cl⁻ concentration (data not shown). This allowed the determination of the k₂: 5.46±0.67 M⁻¹.s⁻¹ (Table 11). This ratio was very low compared to those obtained with other (pseudo)-halides substrates for wt RbMPO.

TABLE 11
Kinetic parameters of wt RbMPO for the NaCl
range at 300 nM of enzyme and 79 μM of H₂O₂

| | Steady state (NaCl range) |
|---|---|
| | kₛₛ/[Cl⁻] (M⁻¹ · s⁻¹) |
| wt RbMPO | 5.46 ± 0.67 |

From the above results, the substrate specificity for wt RbMPO could be determined to be, according the following order: SCN⁻>Br⁻>Cl⁻. Mutants (D202A), (N317M) and (H203A) showed a higher specificity for SCN⁻ than Br⁻, while mutant (H407A) had a greater specificity for Br⁻ than for SCN⁻. The mutants (E316A) and (D202A, E316A) were active only for SCN⁻, while mutant (Q199A) was only active for Br.

Only the native enzyme was studied for the H₂O₂ range: the H₂O₂ range in the presence of SCN⁻ was performed at pseudo-halide saturation, i.e. 5 mM (data not shown). For the halides Cl⁻ and Br⁻, the ranges were not performed at halide saturation but at 500 and 100 mM respectively (data not shown). By applying equation 2 to the kₛₛ values as a function of H₂O₂ concentration, a characteristic profile of wt RbMPO inhibition by H₂O₂ could be observed (data not shown). It must however be noted that the enzyme did not have the same sensitivity to H₂O₂ depending on the halogenated or pseudo halogenated substrate (factor of 10, data not shown). Furthermore, the kₛₛ values were 33 times lower in the presence of Cl⁻ than in the presence of SCN⁻, and 1.4 times lower between Br⁻ and SCN⁻(Table 12). K_{M} values were 0.12±0.04 mM in presence of 5 mM SCN⁻ and 0.23±0.21 mM for 100 mM of Br⁻.

TABLE 12
Kinetic parameters of wt RbMPO at various concentrations of
H₂O₂ and at fixed concentrations of (pseudo)-halogen ions

| | Steady state (H₂O₂ range) | | |
|---|---|---|---|
| wt RbMPO | 267 nM | 267 nM | 1.78 μM |
| Substrate | SCN (5 mM) | Br (100 mM) | Cl (500 mM) |
| K_{Mapp} (mM) | 0.12 ± 0.04 | 0.23 ± 0.21 | 7.64 10⁻³ ± 4.80 10⁻³ |
| k_{catapp} (s⁻¹) | 203.24 ± 45.46 | 113.72 ± 72.11 | 6.87 ± 2.09 |
| k_{catapp}/K_{MappH2O2} | 1.69 10⁶ ± 99.9 10⁵ | 4.94 10⁵ ± 7.65 10⁵ | 8.99 10⁵ ± 8.38 10⁵ |
| (M⁻¹ · s⁻¹) | | | |
| K_{I} (mM) for H₂O₂ | 0.71 ± 0.29 | 0.93 ± 1.22 | 0.006 ± 0.004 |

Thus, the K_{M} in presence of 500 mM Cl⁻ was 7.64 10⁻³±4.86 10⁻³ mM. With this value, a substrate inhibition type fit was then performed to determine the other kinetic parameters of the wt RbMPO under these conditions. The inhibition constant of the enzyme was 0.71±0.29 mM H₂O₂ in the presence of 5 mM SCN⁻ and 0.93±1.22 mM in the presence of Br⁻. For Cl⁻, the Kᵢ value was lower than with other substrates, 0.006±0.004 mM, reflecting an increased sensitivity of the enzyme for this substrate. Interestingly, the catalytic efficiency was quite similar between the three substrates. These results corroborate the findings shown in the previous paragraph with respect to substrate specificity. Wt RbMPO has a better catalytic efficiency on H₂O₂ when coupled with SCN⁻ followed by Cl⁻ and Br⁻ (as the K_{M} of H₂O₂ is lower in the presence of Cl⁻).

The effect of temperature on the activity of the wt RbMPO enzyme was studied by stopped-flow using a range of temperature from 10 to 60° C., with fixed concentrations of halides (SCN⁻: 5 mM with 267 nM RbMPO; Br⁻:500 mM with 267 nM RbMPO; Cl⁻: 500 mM with 1.78 μM RbMPO) and H₂O₂(79 μM). In the presence of SCN⁻, the kₛₛ values were relatively similar from 20 to 60° C. This was also the case in the presence of Cl⁻ and Br⁻ over this same temperature range. See Table 13 below.

TABLE 13
Effect of temperature on the formation of halogenate or pseudo-halogenate compounds by the wt RbMPO.

| | | Temperatures (° C.) | | | | | | |
|---|---|---|---|---|---|---|---|---|
| | | 10 | 20 | 30 | 37 | 40 | 50 | 60 |
| kₛₛ (s⁻¹) | SCN | 5.0 ± 0.01 | 37.6 ± 0.10 | 56.3 ± 0.10 | 63.2 ± 0.11 | 55.5 ± 0.14 | 56.5 ± 0.14 | 67.2 ± 0.22 |
| | Br | 9.5 ± 0.01 | 23.4 ± 0.02 | 24.8 ± 0.02 | 19.5 ± 0.01 | 29.1 ± 0.02 | 21.1 ± 0.01 | 37.8 ± 0.03 |
| | Cl | 0.7 ± 0.001 | 5.6 ± 0.01 | 8.4 ± 0.01 | 9.5 ± 0.02 | 8.3 ± 0.02 | 8.5 ± 0.02 | 10.1 ± 0.03 |

Measurements were carried out at 240, 253 and 289 nm for −OSCN, Tau-NHCl and Tau-NHBr respectively at 37° C. in a 50 mM pH 7.5 sodium phosphate buffer. Each condition had a minimum average of 5 points.

A pH range was also tested in order to characterize the optimal pH for wt RbMPO activity under standard conditions (fixed concentrations of halides namely SCN⁻: 5 mM with 267 nM RbMPO; Br⁻:500 mM with 267 nM RbMPO; Cl⁻: 500 mM with 1.78 M RbMPO) and H₂O_{2 79} μM). The maximum activity for SCN⁻ and Br⁻ was between pH 6 and 7. For Cl, the profile was slightly different with a better enzyme activity between pH 5 and 7. See Table 14 below.

TABLE 14
Effect of pH on the formation of halogenate or pseudo-halogenate compounds by the wt RbMPO.

| | | pH | | | | | | |
|---|---|---|---|---|---|---|---|---|
| | | 4 | 5 | 6 | 7 | 8 | 9 | 10 |
| kₛₛ (s⁻¹) | SCN⁻ | 0.7 ± 0.04 | 5.4 ± 0.02 | 27.0 ± 0.06 | 42.8 ± 0.13 | 20.4 ± 0.03 | 3.1 ± 0.02 | 0 ± 0 |
| | Br⁻ | 0 ± 0 | 1.5 ± 0.004 | 14.5 ± 0.03 | 11.0 ± 0.02 | 4.8 ± 0.01 | 1.3 ± 0.004 | 0.8 ± 0.007 |
| | Cl⁻ | 0 ± 0 | 1.0 ± 0.05 | 1.1 ± 0.01 | 1.6 ± 0.02 | 8.6 10⁻³ ± 1.8 10⁻³ | 6.6 10⁻³ ± 6.2 10⁻⁴ | 3.5 10⁻³ ± 4.2 10⁻⁴ |

Measurements were carried out at 240, 253 and 289 nm for ⁻OSCN, Tau-NHCl, and Tau-NHBr respectively at 37° C. in a 50 mM pH 7.5 sodium phosphate buffer. Each condition had a minimum average of 5 points.

2.5. Pre-steady state kinetics parameters of wt RbMPO

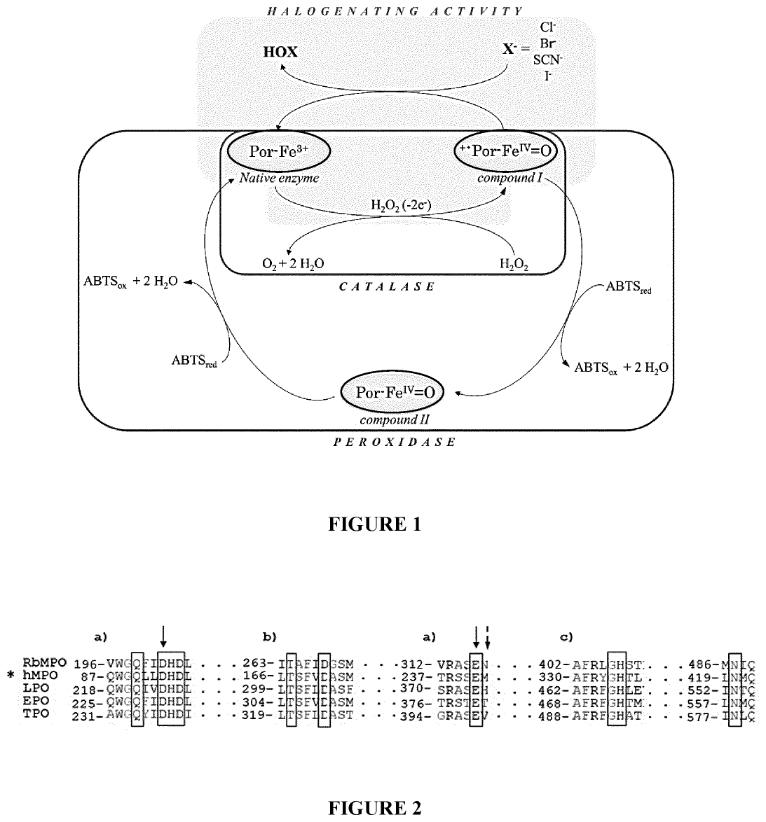

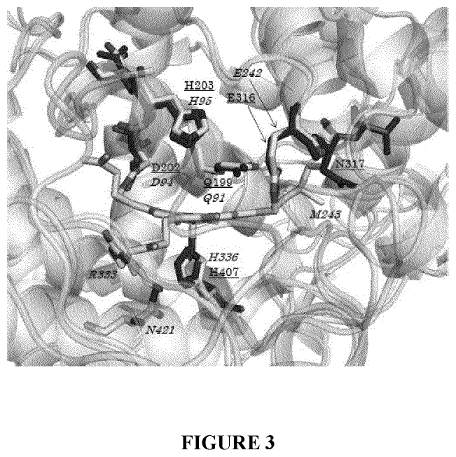

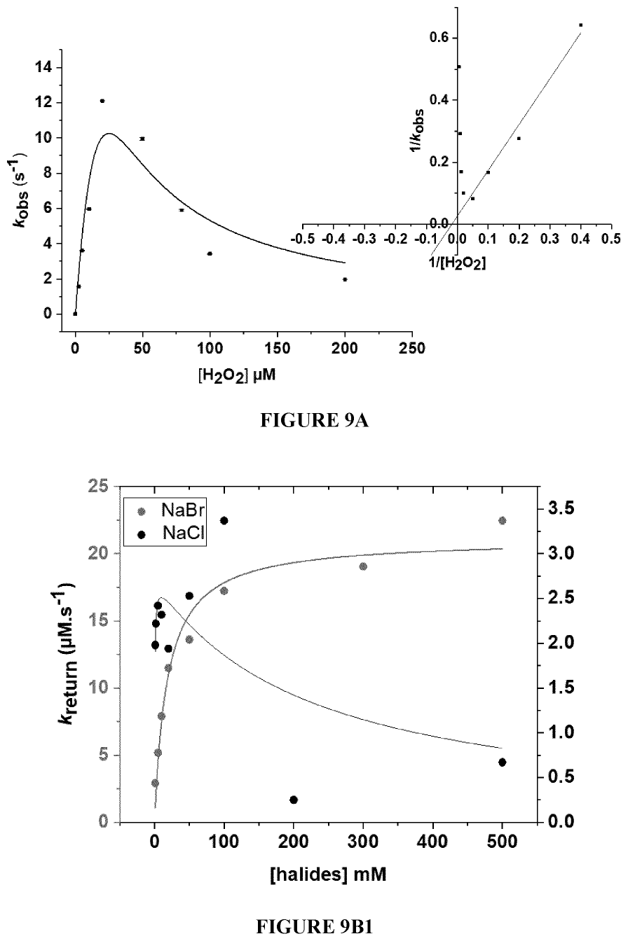

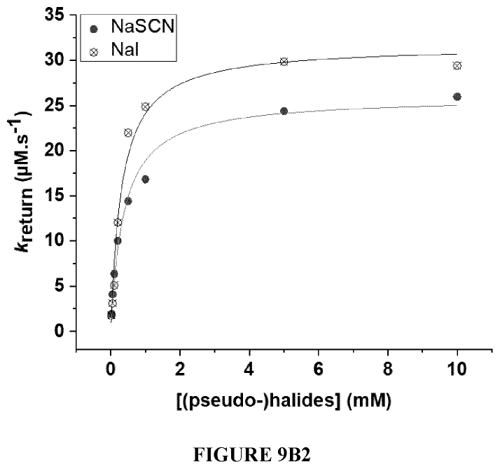

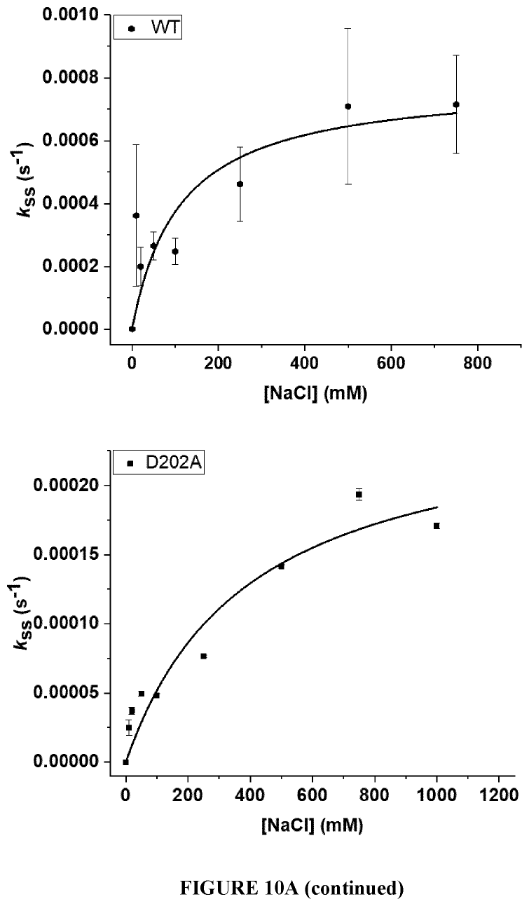

The formation of compound I of wt RbMPO was studied with single turnover conditions by rapid kinetics analysis. The transition of the enzyme from its native form to its compound I form results from the oxidation of heme Fe^{III} to Fe^{IV} (FIG. 1). The heme Soret band is at 412 nm for the native Fe^{III} iron form, which is why the decrease in absorbance was followed at this particular wavelength as a function of time. The rate constant was measured using the A₄₁₂ₙₘ curves as a function of time (data not shown). By applying Eq.4 to the exponential part of each kinetic, the values of Tau (s) were determined and the k_{obs} (s⁻¹) were calculated. The k_{obs} values as a function of H₂O₂ concentration showed a hyperbolic pattern with an inhibition by excess of substrate (FIG. 9A). It was from a Kᵢ value of 10.90+3.00 mM that the transition rate of the wt RbMPO from its native state to compound I decreased (Table 15). The ratio k_{obsmax}/K_{D} at 1.00 10⁶±1.30 10⁶ M⁻¹.s⁻¹ showed a significant catalytic efficiency of the wt RbMPO for the formation of compound I in the presence of H₂O₂.

TABLE 15
Catalytic parameters of compound I formation

| | wt RbMPO: 1.78 μM |
|---|---|
| K_{D} (μM) | 56.89 ± 20.13 |
| k_{obsmax} (s⁻¹) | 57.09 ± 8.60 |
| k_{obsmax}/K_{D} (M⁻¹ · s⁻¹) | 1.00 10⁶ ± 5.06 10⁵ |
| Ki (μM) | 10.90 ± 3.00 |

The return of the enzyme to its native state was studied by the impact of increasing concentrations of (pseudo)-halides substrates on compound I (FIG. 9B). The kinetics at 412 nm enabled to highlight the formation of compound I with an exponential decrease in absorbance followed by recovery to the initial A₄₁2m value when there was not enough H₂O₂ (data not shown). It is the time difference (s) between the two characteristic steps that allowed the calculation of the values of the Vᵣₑₜᵤᵣₙ(μM⁻¹.s⁻¹) values:

$${V}_{return} = \frac{[ {H}_{2} {O}_{2} ]}{{time}_{return}}$$

The SCN, Br⁻ and I⁻ ranges had one thing in common: the return to the native state of wt RbMPO was characterised by the saturation of the enzyme by the (pseudo)-halide substrate (FIG. 9B1 and B2). In the case of Cl⁻, the curve profile showed inhibition with excess of NaCl, with a Kᵢ 217.05±227.85 mM (FIG. 9B1 and Table 16). However, the Vᵣₑₜᵤᵣₙ/K_{1/2} ratio that reflects the catalytic efficiency of the enzyme to return to its native state was greater for Cl⁻ than for Br⁻: 5169±4872 M⁻¹.s⁻¹ and 607.77±161.51 M⁻¹.s⁻¹ respectively (Table 16). This experiment also showed that I⁻ was a preferential substrate for wt RbMPO because in its presence, the enzyme returned more efficiently to its native state with a Vᵣₑₜᵤᵣₙ/K_{1/2} ratio of 5.37 10⁴±1.07 10⁴ M⁻¹.s⁻¹ (Table 16). In the presence of increasing concentrations of SCN⁻, the return to the native state was also very efficient with a rate of 3.69 10⁴±6.36 10³ M⁻¹.s⁻¹ but lower than with I⁻ (Table 16). The pre-steady state study therefore consolidates the previous conclusions regarding substrate specificity. It also shows that the substrate influences the return rate to the native state of the wt RbMPO, which confirms the previous findings, and substrate specificity as follow: I⁻>SCN⁻>Br⁻ Cl⁻. It could also be concluded that pseudo-(halides) do not influence the formation of compound I except for I⁻ where the rate of formation of compound I increased with concentration (data not shown).

TABLE 16
Catalytic parameters of wt RbMPO return to its native state

| | Return to native state | | | |
|---|---|---|---|---|
| wt RbMPO | 1.9 μM | | | |
| Substrate 1 | Br⁻ | Cl⁻ | SCN⁻ | I⁻ |
| (range) | | | | |
| Substrate 2 | H₂O₂: 20 μM | | | |
| K_{1/2} (mM) | 18.28 ± 3.92 | 0.26 ± 0.21 | 0.37 ± 0.05 | 0.31 ± 0.05 |
| k_{return max} | 21.11 ± 1.09 | 2.61 ± 0.37 | 25.94 ± 0.97 | 31.65 ± 1.23 |
| (μM · s⁻¹) | | | | |
| wt RbMPO | 1.9 μM | | | |
| Substrate 1 | Br⁻ | Cl⁻ | SCN⁻ | I⁻ |
| (range) | | | | |
| kᵣₑₜᵤᵣₙ/K_{1/2} | 607.77 ± 161.51 | 5169 ± 4872 | 3.69 10⁴ ± 6.36 10³ | 5.37 10⁴ ± 1.07 10⁴ |
| (M⁻¹ · s⁻¹) | | | | |
| Vᵣₑₜᵤᵣₙ (s⁻¹) | 11.11 ± 0.57 | 1.37 ± 0.19 | 13.65 ± 0.51 | 16.66 ± 0.65 |
| (Sᵣₑₜᵤᵣₙ/[enzyme]) | | | | |
| Kᵢ (mM) | n.a. | 217.05 ± 227.85 | n.a. | n.a. |

(n.a.: not available).

### 2.6. Chlorination Activity of Wt and Mutated RbMPOs

The kinetic parameters of wt RbMPO and mutants thereof for Cl⁻ were determined using APF, and was complementary to the stopped-flow experiment. Enzyme substrate ranges were performed in both NaCl and H₂O₂. The slopes were measured at the onset of the reaction in order to be in initial velocity. From these values, it was possible to calculate the kₛₛ with the following equation:

$${k}_{ss} = \frac{slopes ( FI \cdot {s}^{- 1} )}{slopes ( FI \cdot {pmoles}^{- 1} )} \frac{1}{pmoles of RbMPOs}$$

where slopes in FI.pmoles⁻¹ corresponds to the slope of the fluorescein standard range: 2.95 10⁶⁺1.13 10⁵.

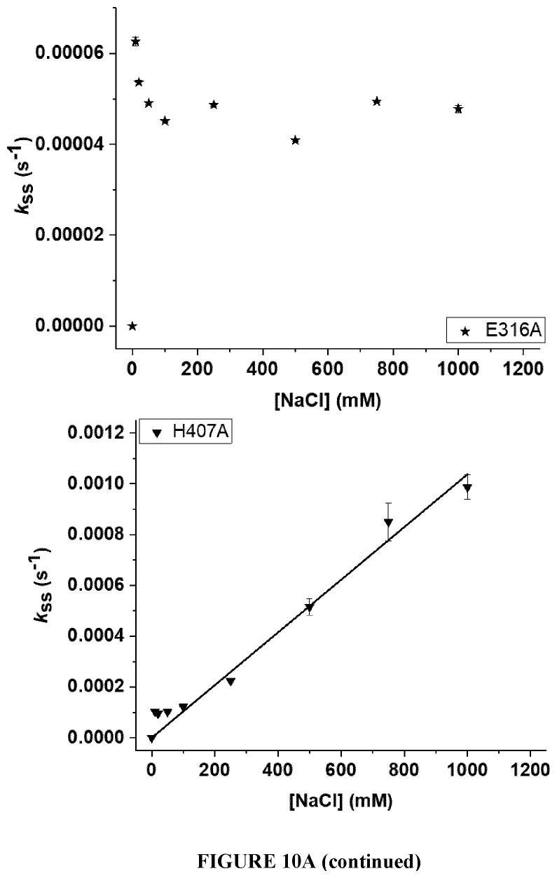

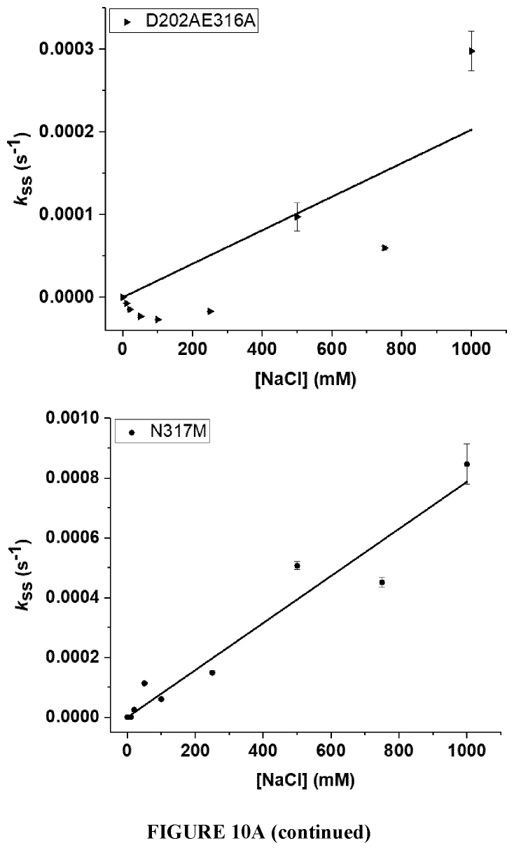

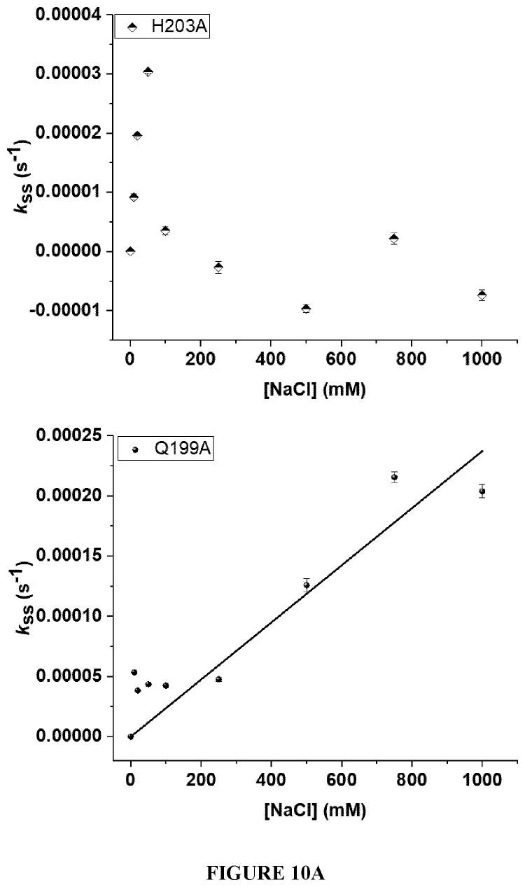

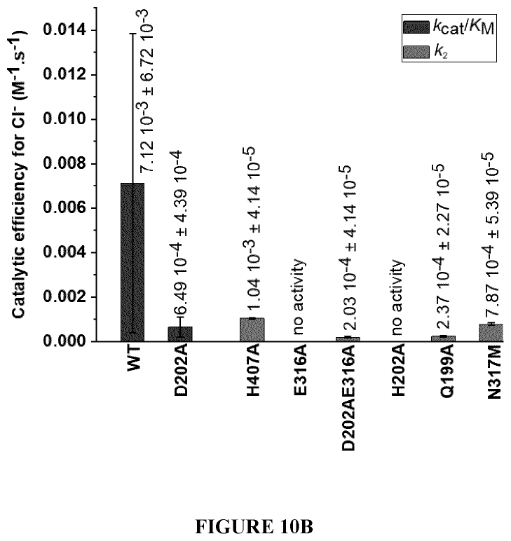

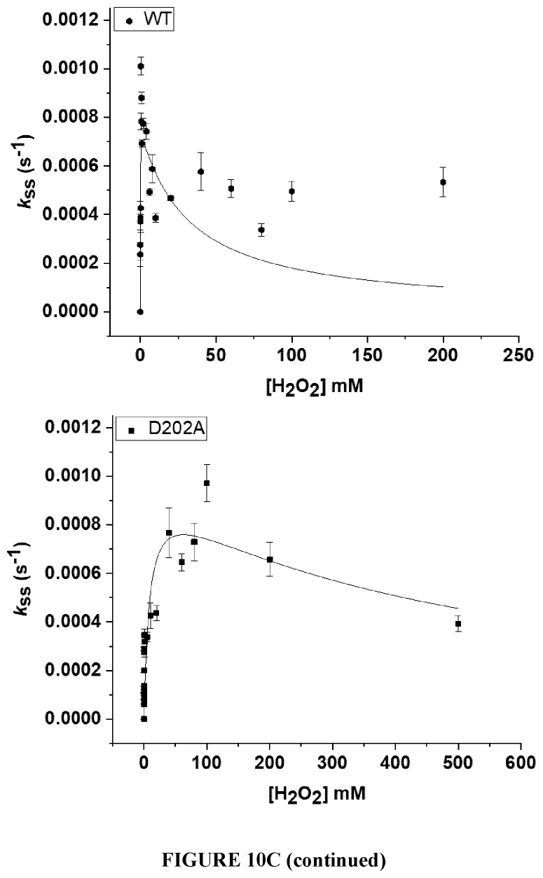

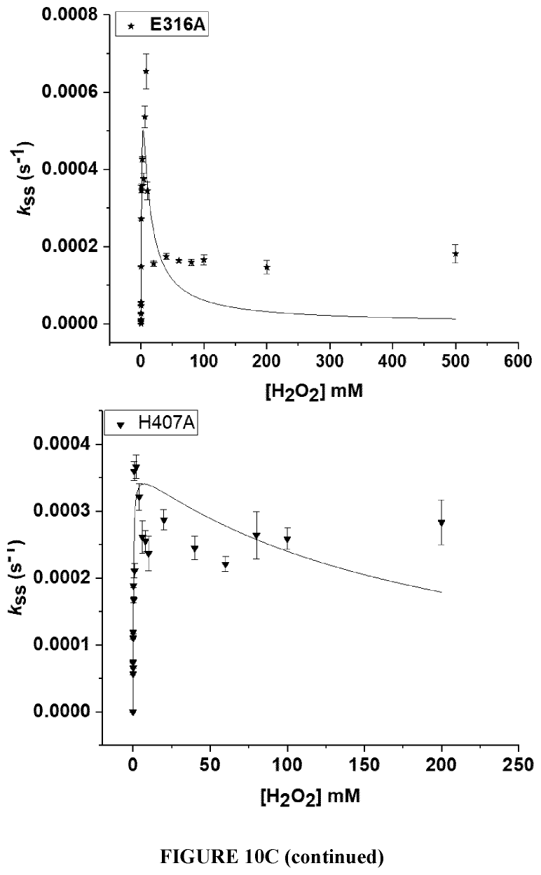

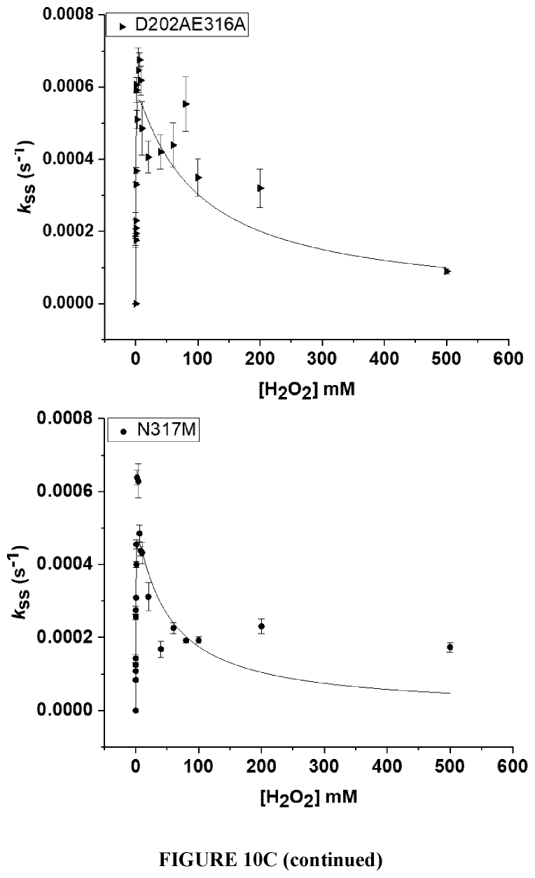

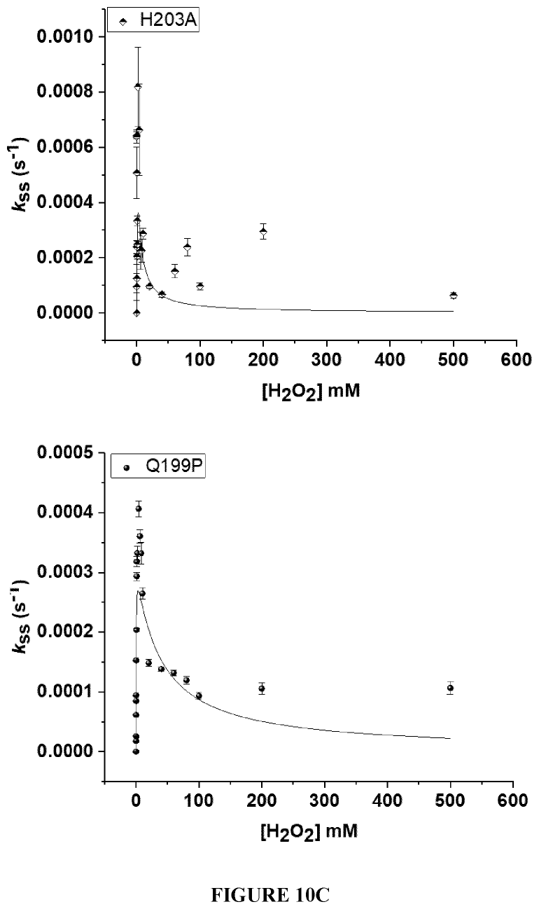

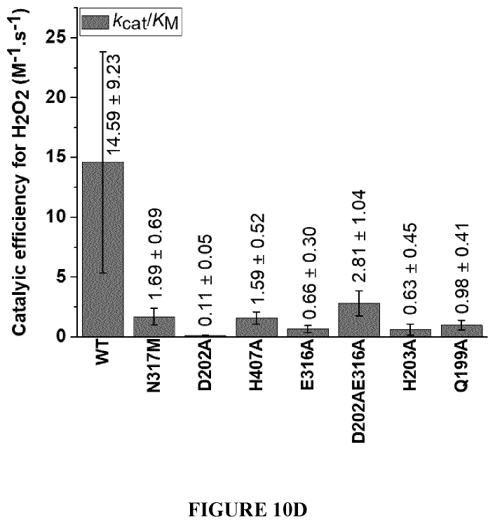

Increasing the NaCl concentration in the assay lead to a Michaëlis-Menten curves profile for (D202A) and wt RbMPOs and to a linear increase in the kₛₛ values for the other mutants (FIG. 10A). These results allowed the calculation of k_{cat}/K_{M} in M⁻¹.s⁻¹. The wt RbMPO had the highest catalytic efficiency in Cl⁻ with 7.12 10⁻³ 6.72 10⁻³ M⁻¹.s⁻¹ (FIGS. 10A and 10B; Table 17).

The mutant (D202A) had Cl⁻ efficiency 7-fold lower than wt RbMPO (1.04 10⁻³ 4.14 10⁻⁵ M⁻¹.s⁻¹) (Table 17). (N317M), (D202A), (Q199A) and (D202A, E316A) had the following k₂ values: 7.87 10⁻⁴ 5.39 10-5 M⁻¹.s⁻¹; 6.49 10⁻⁴ 4.39 10⁻⁴ M⁻¹.s⁻¹; 2.37 10⁻⁴ 2.27 10⁻⁵ M⁻¹.s⁻¹; 2.03 10⁻⁴ 4.14 10⁻⁵ M⁻¹.s⁻¹ (Table 17).The two mutants (E316A) and (H203A) had kₛₛ values with sparse data; no catalytic constant could be determined in these two cases. These results show that a mutation in the active site leads to changes in the efficiency of the enzyme for Cl, suggesting that all these amino acids are important in the activity of RbMPO.

TABLE 17
Kinetics parameters of chlorination activity
of wt and mutated RbMPOs (NaCl range)

| Enzymes | K_{M} (mM) | k_{cat} (s⁻¹) | k_{cat}/K_{M} (M⁻¹ · s⁻¹) |
|---|---|---|---|
| wt RbMPO | 110.75 ± 80.03 | 7.89 10⁻⁴ ± 1.74 10⁻⁴ | 7.12 10⁻³ + 6.72 10⁻³ |
| D202A | 394.26 ± 188.90 | 2.56 10⁻⁴ ± 5.06 10⁻⁵ | 6.49 10⁻⁴ ± 4.39 10⁻⁴ |

| | k₂ (M⁻¹ · s⁻¹) |
|---|---|
| H407A | 1.04 10⁻³ ± 4.14 10⁻⁵ |
| N317M | 7.87 10⁻⁴ ± 5.39 10⁻⁵ |
| D202A, E316A | 2.03 10⁻⁴ ± 4.14 10⁻⁵ |
| E316A | scatter plot |
| H203A | scatter plot |
| Q199A | 2.37 10⁻⁴ ± 2.27 10⁻⁵ |

For the H₂O₂ range, the kₛₛ values of the enzymes had a very different profile compared to the NaCl range (FIG. 10C). The substrate inhibition equation was applicable for each enzyme. Wt RbMPO had the highest k_{cat}/K_{M} (14.59+9.23 M⁻¹.s⁻¹) ratio, indicating a higher efficiency of this form for H₂O₂ and the lowest K_{M} (0.052±0.028 mM) (FIGS. 10c and 10D; Table 18). Furthermore, the native form of the enzyme was sensitive to H₂O₂ as it was inhibited from 31.54±20.0 mM (Table 18). The mutant (D202A, E316A) had 5-fold lower efficiency for H₂O₂ than wt RbMPO. The mutants (N317M), (H407A) and (Q199A) had more and less the same ratio values: 1.69±0.69 M⁻¹.s¹, 1.59±0.52 M⁻¹.s⁻¹ and 1.35±0.37 M⁻¹.s⁻¹ respectively (Table 18). The difference between these three mutants was that H407A showed inhibition by H₂O₂ with a Kᵢ 190.98±69.75 mM; this form of enzyme form was less sensitive than the two other mutants to H₂O₂. Finally, mutants (H203A), (E316A) and (D202A) had very low k_{cat}/K_{M}ratio (0.63±0.45 mM, 0.66±0.30 mM and 0.11±0.05 mM) due to high K_{M} values (1.31±0.72 mM, 1.49±0.37 mM and 8.77+3.19 mM respectively) (Table 18). For this substrate, mutations in the active site altered the efficiency and sensitivity of the enzyme to H₂O₂.

TABLE 18
Kinetics parameters of chlorination activity of wt and mutated RbMPOs (H₂O₂ range)

| | | K_{M} H₂O₂ | k_{cat}/K_{M} H₂O₂ | |
|---|---|---|---|---|
| Enzymes | k_{cat} (s⁻¹) | (mM) | (M⁻¹ · s⁻¹) | Kᵢ H₂O₂ (mM) |
| wt RbMPO | 7.59 10⁻⁴ ± 7.11 10⁻⁵ | 0.052 ± 0.028 | 14.59 ± 9.23 | 31.17 ± 12.79 |
| N317M | 5.40 10⁻⁴ ± 6.76 10⁻⁵ | 0.32 ± 0.09 | 1.69 ± 0.69 | 48.10 ± 15.44 |
| D202A | 9.71 10⁻⁴ ± 7.61 10⁻⁵ | 8.77 ± 3.19 | 0.11 ± 0.05 | 449.58 ± 197.99 |
| H407A | 3.66 10⁻⁴ ± 1.76 10⁻⁵ | 0.27 ± 0.09 | 1.59 ± 0.52 | 190.98 ± 69.75 |
| E316A | 9.82 10⁻⁴ ± 2.08 10⁻⁴ | 1.49 ± 0.37 | 0.66 ± 0.30 | 6.63 ± 2.53 |
| D202A, E316A | 6.19 10⁻⁴ ± 6.06 10⁻⁵ | 0.22 ± 0.06 | 2.81 ± 1.04 | 96.33 ± 31.94 |
| H203A | 8.19 10⁻⁴ ± 1.44 10⁻⁴ | 1.31 ± 0.72 | 0.63 ± 0.45 | 3.35 ± 2.24 |
| Q199A | 4.80 10⁻⁴ ± 5.34 10⁻⁵ | 0.49 ± 0.15 | 0.98 ± 0.41 | 18.28 ± 5.34 |

### 2.7. Catalase Activity of Wt RbMPO

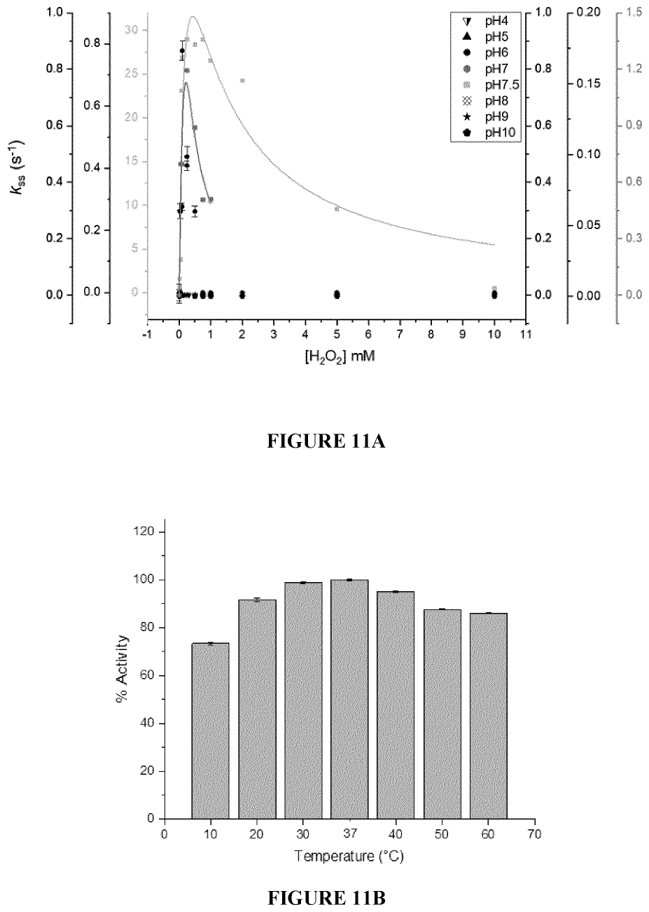

The measurement of the decrease in absorbance at 240 nm allowed the characterization of the catalase activity of the wt RbMPO (FIG. 11).

At pH4, 5, 8 and 9, the enzyme showed no detectable catalase activity with all kₛₛ values equal to 0 (FIG. 11A and Table 19). At pH6 and pH10, the kₛₛ values were so close to 0 that it the catalase activity was considered negligible. By contrast, the wt RbMPO showed significant catalase activity at pH7.5, with the curve kₛₛ versus H₂O₂ concentration showing a substrate inhibition profile of the enzyme with a k_{cat}/K_{M} ratio of 3.33 10⁵+3.36 10⁵ which is quite significant (Table 19). The curve profile for pH7 was similar to that of pH7.5 up to 1 mM H₂O₂, but with and kₛₛ values 24 times lower on average. In addition, the k_{cat}/K_{M} ratio was 4.05 10⁴ 6.20 10⁴ (Table 19).

TABLE 19
Catalase activity of wt RbMPO at pH 7 and pH 7.5

| | pH 7 | pH 7.5 |
|---|---|---|
| K_{M} (mM) | 0.04 ± 0.04 | 0.17 ± 0.11 |
| k_{cat} (s⁻¹) | 1.62 ± 0.86 | 56.64 ± 20.62 |
| k_{cat}/K_{M} (M⁻¹ · s⁻¹) | 4.05 10⁴ ± 6.20 10⁴ | 3.33 10⁵ ± 3.36 10⁵ |
| Kᵢ (mM) | 0.54 ± 0.57 | 1.07 ± 0.7 |

Between 20° C. and 40° C., the enzyme kept more than 90% of catalase activity; from about 50° C. to about 60° C., the enzyme kept more than 80% of catalase activity; and at about 10° C., the enzyme kept more than 70% of catalase activity (FIG. 11B and Table 20).

TABLE 20
Catalase activity of wt RbMPO at a temperature
ranging from 10° C. to 60 C. °

| Temperatures | kₛₛ (s⁻¹) |
|---|---|
| 10° C. | 2.04 ± 0.01 |
| 20° C. | 2.55 ± 0.02 |
| 30° C. | 2.75 ± 0.01 |
| 37° | 2.78 ± 0.01 |
| 40° C. | 2.64 ± 0.01 |
| 50° C. | 2.43 ± 0.01 |
| 60° C. | 2.39 ± 0.01 |

### 2.8. Peroxidase Activity of Wt RbMPO

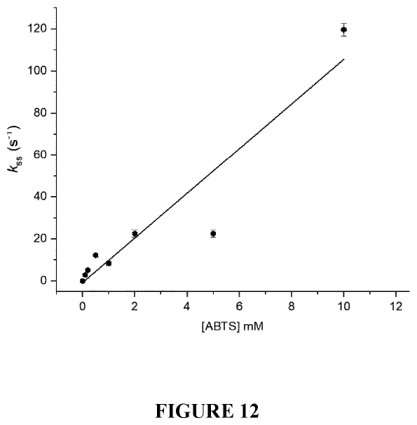

kₛₛ values were found to increase linearly with ABTS concentration with a k₂ value of 10620 1510 M⁻¹.s⁻¹ (FIG. 12).

### 2.9. Stabilization Conditions for Storage of Wt RbMPO

The value obtained at TO is defined as 100% of the enzymatic (MPO) activity at the temperature tested and all the other activities are calculated from this TO timepoint.

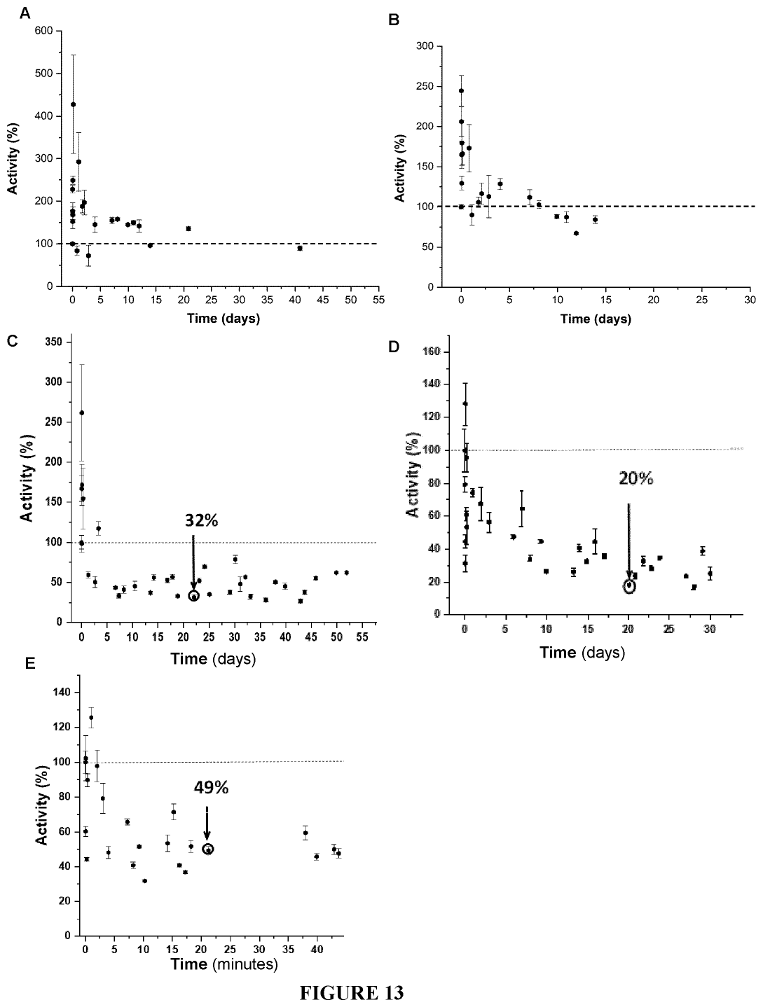

For the wt RbMPO stored in the 50 mM Tris buffer pH 7.5, after a few minutes, the enzyme showed a higher myeloperoxidase activity at both temperatures (4° C. and 37° C.); this explains why activity percentages greater than 100% were observed at the start of the measurements. After 20 days at 4° C. in the Tris buffer, the enzyme was still 100% active (FIG. 13A). At 37° C., the activity remained stable, at about 80% after about 15 days (FIG. 13B). The myeloperoxidase activity of wt RbMPO remained stable (very close to 100%) after being stored in the presence of 50 mM Tris buffer pH7.5, for up to 30 days either at 4° C. or at 37° C.

By contrast, the wt RbMPO stored in the 50 mM NaPi buffer pH 7.5 lost rapidly its myeloperoxidase activity. While the myeloperoxidase activity rapidly increased (over 100%) in the first few minutes, the activity of the enzyme quickly decreased overtime. By comparison to the Tris buffer storage conditions, the enzyme activity reached only 32% after storage at 4° C. for about 20 days (FIG. 13C), and 30% after storage at 37° C. for about 15 days (FIG. 13D)—even decreasing to about 20% after 20 days at 37° C.

Likewise, the myeloperoxidase activity of wt RbMPO stored in the PBS buffer pH 6.6 rapidly decreased. Again, the activity increased (over 100%) in the first couple of minutes, only to sharply fall thereafter, by 50% (FIG. 13E).

Storing the wt RbMPO in a 50 mM Tris buffer pH7.5, at either 4° C. or 37° C., is thus optimal to ensure preserving its efficiency over time.

### 2.10. Microbicidal Activity

By measuring bacterial kinetics in a microplate

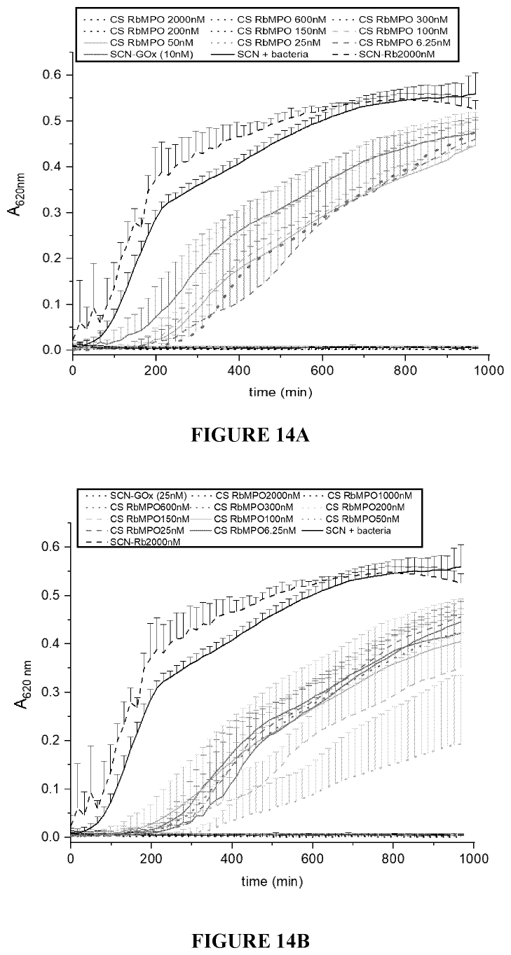

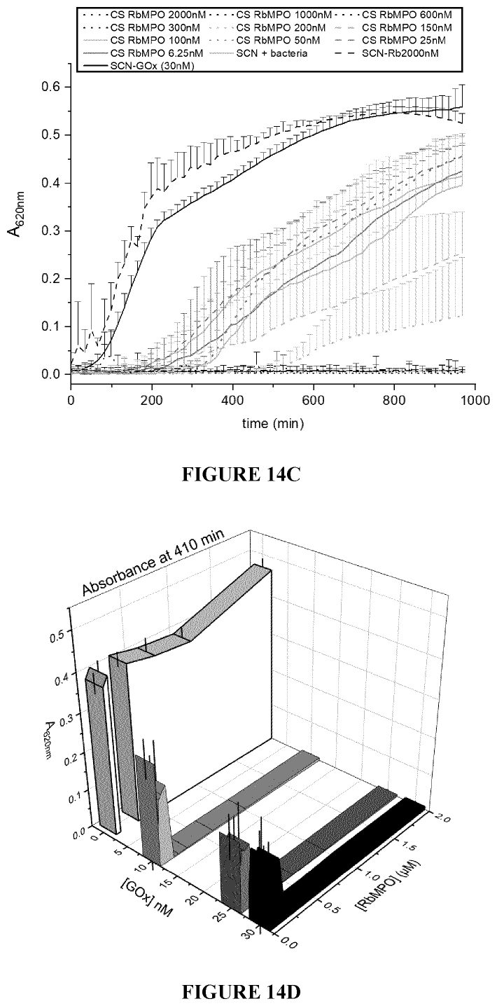

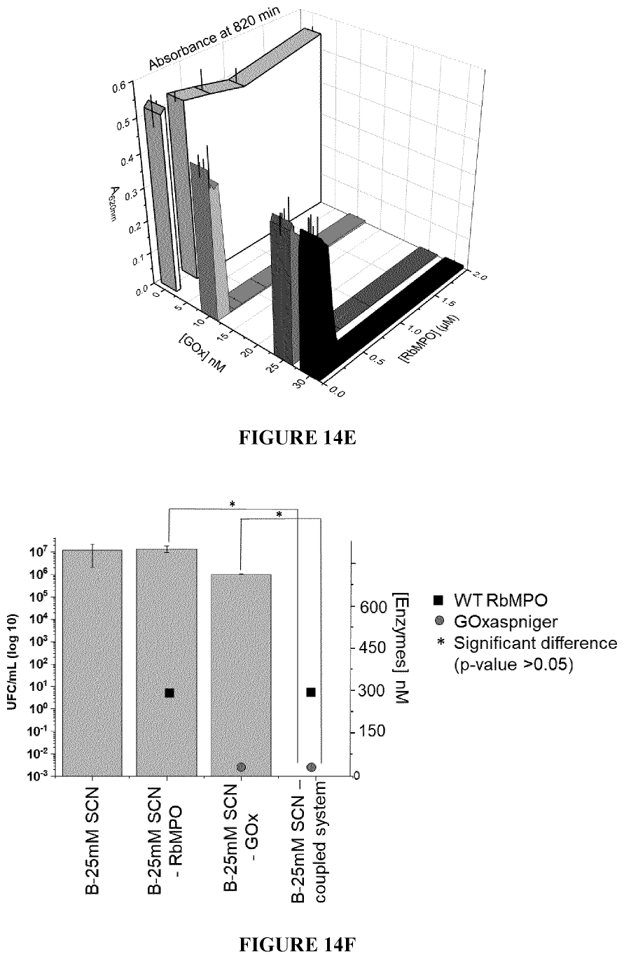

FIG. 14B shows the antimicrobial effect using different concentration ranges of wt RbMPO in the presence of 25 nM GOx_{aspniger} and 25 mM of NaSCN, at pH 7.4. For wt RbMPO concentrations ranging from 0 nM to 100 nM, the curves overlapped and showed a slowing of bacterial growth. For concentrations of 150 nM and 200 nM of enzyme (ratios 6:1 and 8:1 in wt RbMPO: GOx_{aspniger}), the decrease in A₆₂₀ₙₘ values was even more pronounced. From 300 nM of wt RbMPO, there was a total inhibition of bacteria growth. Therefore, a minimum ratio of 12:1 (wt RbMPO: GOx_{aspniger}) was necessary to lead to this 100% bactericidal effect over 16h of kinetic.

When different concentration ranges of wt RbMPO were used in presence of 10 nM GOx_{aspniger}, a total inhibition of bacterial growth was observed from 150 nM to 2000 nM wt RbMPO (FIG. 14A). Therefore, at least 15 times more wt RbMPO than GOx_{aspniger} was required to have a 100% bactericidal effect.

When different concentration ranges of wt RbMPO were used in presence of 30 nM of GOx_{aspniger} and 25 mM of NaSCN, still at pH7.4, a ratio of at least 10:1 (wt RbMPO: GOx_{aspniger}) was required to inhibit 100% of bacterial growth over 16h (FIG. 14C).

As the shown in the kinetics, the absorbance at 620 nm was equal to 0 (hence, 100% inhibition of bacterial growth) with 150 nM wt RbMPO and 10 nM GOx_{aspniger} (15:1 ratio) or with 300 nM wt RbMPO for the 25 nM and 30 nM GOx_{aspniger} conditions (12:1 and 10:1 ratios, respectively) (FIGS. 14A to E).

In all these experiments, the pH used was at 7.4. Yet, it was clearly the halogenation activity that was favoured and not the catalase activity. The use of 25 mM SCN⁻ in the experiments seemed to direct wt RbMPO towards its halogenation activity.
By counting colonies on Petri dishes

This method is commonly used in pharmacopoeia to test the effect of antibiotics on microorganisms. It was used herein to also demonstrate the bactericidal effect of wt RbMPO The results are displayed in FIG. 14F.

To this end, E. coli ATCC 25922 bacteria were incubated for 1 h in solutions containing the enzymes and substrate required to produce −OSCN: 25 mM of NaSCN, 25 nM of GOx_{aspniger} and 300 nM of wt RbMPO at pH7.4.

As expected, in the absence of hydrogen peroxide or source of hydrogen peroxide, there was no significant difference between the number of colonies on the “25 mM SCN” control (12,150,000±10,111,627 CFU/mL) and the following conditions: 25 mM SCN⁻ and 300 nM wt RbMPO (13,850,000±4,454,773 CFU/mL) with a p-value of 0.85. Likewise, the same result was observed between the “bacteria+SCN” control versus the pseudo-halogenated substrate in the presence of 25 nM GOx_{aspniger} (1,015,000±21,213 CFU/mL) with a p-value of 0.26, due to the absence of the myeloperoxidase. If the two “enzyme only” controls were compared to each other, then the p-value of 0.055 was not considered significant. It was in the presence of the coupled system 25 nM GOx_{aspniger} and 300 nM RbMPO (with 25 mM SCN⁻) that an inhibition of bacterial growth was observed. As in the 96-well plate experiment, 25 mM SCN⁻ in the presence of 25 nM GOx_{aspiniger} and 300 nM wt RbMPO was sufficient to kill 100% of the bacteria (p-values of 0.0002 with “GOx_{aspniger} alone” and 0.048 “wt RbMPO alone”). Here, only a one-hour incubation between the 1.10⁶ CFU/mL bacterial dilution and the coupled system solution was sufficient to fully inhibit bacterial growth.

### 3. Discussion

In the present study, a robust production protocol, two-step chromatographic purification and reconstitution of RbMPO had been established, allowing the production of approximately 30 mg of pure enzyme per 2L of culture. Pure wt RbMPO was predominantly in monomeric form as shown by DLS and size exclusion chromatography experiments (Superose 12).

The different reconstitution tests of the enzyme in its holoenzyme form showed that the optimum condition was at pH7.5 in 50 mM Tris for 24 hours in the presence of a large excess of heme. A complementary study of the fluorescence quenching of RbMPO showed that the enzyme has one heme per monomer. A slow conformational rearrangement was also observed from time TO*. Monitoring this rearrangement in the presence of increasing heme concentrations indicated an absence of saturation over the concentration ranges tested. Only the results obtained for the mutant (D202A, E316A) allowed the determination of a K_{D} of 10.87+6.14 μM for heme. In the literature, the formation of covalent bonds with heme is described as autocatalytic in the presence of H₂O₂. Here, the reconstitution assays were performed in the absence of H₂O₂ but differences in rearrangement efficiency were measured between the wild type and mutated enzymes. In the absence of the amino acids D202 and E316, reconstitution appeared to be very efficient with a k_{obsmax}/Kₐₚₚ of 2.57 10⁴±2.00 10⁴ M⁻¹.s⁻¹. One hypothesis would be the absence of the autocatalytic reaction of forming two covalent bonds with heme, which would contribute to a better reconstitution efficiency.

The chlorination activity of wt RbMPO was studied in the presence of increasing ABAH concentrations. The results showed that the enzyme is inhibited by this molecule. However, further studies by monitoring the fluorescence of tryptophan of the different RbMPOs showed that, on the holo form, increasing concentrations of ABAH lead to a recovery of the signal corresponding to the signal of the enzyme in the apo form. It was thus hypothesized that ABAH degrades RbMPOs at very high concentrations, by causing the release of heme in the absence of H₂O₂. There was therefore a direct correlation between the inhibition of RbMPO activity and the release of heme from the active site. These results support the findings that the active sites of hMPO and RbMPO are relatively similar.

Determination of stead-state catalytic constants showed that wt RbMPO has the same (pseudo)-halogenated substrate specificity as hMPO, i.e.: SCN⁻ (1.12.10⁵±3.11.10⁴ M⁻¹.s⁻¹)>Br⁻(576.96±172.22 M⁻¹.s⁻¹)>Cl⁻ (4.48±0.64 M⁻¹.s⁻¹). The active site mutants (N317M), (D202A) and (H203A) showed a higher efficiency with SCN⁻ than with Br, like the wt RbMPO. By contrast, for the mutant (H407A), Br⁻ bound more easily in the active site. The mutant (Q199A) was only active in Br⁻ while the mutants (D202A, E316A) and (E316A) mutants were only active in SCN. The amino acid substitution D202, which is thought to be responsible for the formation of a covalent bond between the active site and the heme, did not change the substrate specificity. However, in the case of the E316A substitution (expected to be responsible for the 2^{nd} covalent bond), the enzyme became inactive for Br⁻. The double substitution (D202A, E316A) also resulted in the inactivity of RbMPO for Br⁻. Residues (Q199) and (H203) form the proximal cavity of the heme and the latter corresponds to the Br⁻ binding site. Their replacement by an alanine did not alter the substrate specificity except for Q199A, which became inactive in SCN. The (H407A) substitution reversed the substrate specificity of the enzyme due to a lower K_{M} for Br⁻ than for SCN. This residue was responsible for the curvature of the heme and its replacement by an alanine should result in a change of this curvature, which should probably make the Br⁻ binding pocket in the active site more accessible.

The effects of temperature and pH on the activity of the enzyme were studied separately under standard stopped-flow conditions. Wt RbMPO remained effective for its three (pseudo)-halogenated substrates at elevated temperatures (up to 60° c.). The study of different pH values on the enzymatic activity indicated that the optimal condition is at pH6 in the presence of Br⁻and at pH7 in the presence of Cl⁻ and SCN.

The results of the chlorination activity of wt RbMPO with the stopped-flow versus the plate reader showed that the kₛₛ values differed by a factor 6000 between the two experiments. These divergent results are probably due to the APF probe, which can only be used at pH7.5, which is precisely the pKa value of HOCl. At this pH, there was therefore theoretically 50% of the ⁻OCl form and 50% of the HOCl form. Moreover, both tests for chlorination activity were indirect.

For all forms of RbMPO investigated (wt and mutated), H₂O₂(in the presence of Cl⁻) resulted in an inhibition of the enzyme. The sensitivity of wt RbMPO to H₂O₂ differed depending on the (pseudo)-halogenated substrate used: it had a lower tolerance in the presence of Cl⁻ (Kᵢ=0.006±0.004 mM) followed by SCN⁻ (Kᵢ=0.71±0.29 mM) and finally Br⁻ (Kᵢ=0.93±1.22 mM).

Wt RbMPO also displays a catalase activity, which was observed at pH 7 and 7.5, with k_{cat}/K_{M} of 4.05 10⁴±6.20 10⁴ M⁻¹.s⁻¹ and 3.33 10⁵±1.21 10⁵ M⁻¹.s⁻¹ respectively. This catalase activity was thus 10-fold more efficient at pH 7.5 than pH 7. The specificity represented by the ratio k_{cat}/K_{M} shows that the enzyme is very efficient. Moreover, wt RbMPO displayed a catalase activity from about 10° C. to about 60° C. More than 70% of the activity was maintained at 10° C., more than 80% of activity was maintained at 50° C. and 60° C. and more than 90% of activity was maintained from 20° C. to 40° C.

Concerning the peroxidase activity, the wt RbMPO is also very efficient. It showed a linear dependence of the kₛₛ towards the concentration of ABTS; that is why a k₂ was determined instead of k_{cat}/K_{M}. Wt RbMPO had a high peroxidase activity at pH5.5 with a k₂ of 10620±1510 M¹.s⁻¹.

The results related to chlorination activity of RbMPOs still allowed to draw the following conclusions. The substitutions of the active site residues mainly affected the efficiency of the enzyme for H₂O₂ by strongly decreasing it. As for the Cl⁻ efficiency, it was not drastically modified except for mutant (H407A), which was 4 times more active than the wt form and for the (E316A) and (H203A) mutants whose kₛₛ values did not vary with increasing NaCl concentrations. The (N317M) substitution did not affect the chlorination activity. Contrary to hMPO, this methionine in position 317 did not favour chlorination activity, but the question of the formation of a third covalent bond remains.

The pre-steady state study showed that I⁻ is also a substrate for the enzyme.

Storage of the wt RbMPO in a 50 mM Tris buffer pH7.5, at either 4° C. or 37° C. was identified as optimal conditions for preserving the enzymatic efficiency over time.

The bactericidal experiments provided a proof-of-concept of the antibacterial composition of the invention on the E. coli strain ATCC 25922, using SCN⁻ as a substrate. It was at 25 mM SCN (pH 7.4), between 10 and 30 nM GOx_{aspniger} and ideally from 300 nM wt RbMPO that a 100% bactericidal effect could be observed.

Counting of surviving colonies after 1 h of incubation in an enzymatic solution in the presence of 300 nM wt RbMPO, 25 nM GOx_{aspniger} and 25 mM SCN, confirmed the results observed in kinetics at 620 nm. At these concentrations, SCN⁻ and GOx_{aspniger} promoted a halogenation activity of wt RbMPO, resulting in complete bacterial death.

### 4. Conclusion

In the present study, a new peroxidase was extensively characterized from biochemical and enzymatic approaches. RbMPO was purified from inclusion bodies and reconstituted with heme. RbMPO possesses several activities as shown in FIG. 1: it catalyzes the formation of (pseudo-)hypo halogenates from I⁻, SCN, Br⁻ and Cl⁻ and also the dismutation of H₂O₂ and peroxide oxidation. Key amino acids of the active site were mutated to validate the model and understand the specificity for these substrates. A bactericidal effect of wt RbMPO was further demonstrated. A particular composition which stabilizes the bacterial heme peroxidase, and is thus particularly suited for its storage, was also identified.

1-25. (canceled)

26. An isolated polypeptide comprising an amino acid sequence having at least 95% sequence identity to SEQ ID NO: 1, wherein the polypeptide has a myeloperoxidase-catalase activity.

27. The polypeptide according to claim 26, wherein said polypeptide is in the form of a holoenzyme.

28. An isolated nucleic acid encoding the polypeptide according to claim 26.

29. The isolated nucleic acid according to claim 28, said nucleic acid comprising a nucleotide sequence having at least 95% sequence identity to SEQ ID NO: 2.

30. A vector comprising the isolated nucleic acid according to claim 28.

31. A host cell comprising the vector according to claim 30.

32. A method for obtaining an isolated polypeptide having a myeloperoxidase-catalase activity, comprising at least the steps of:
a) culturing in a medium a host cell according to claim 31, under conditions suitable for the expression of the polypeptide; and
b) recovering said polypeptide.

33. A composition for stabilizing a polypeptide having a myeloperoxidase-catalase activity, said composition comprising the polypeptide according to claim 26 and a stabilizing agent.

34. The composition according to claim 33, wherein the stabilizing agent is Tris buffer.

35. An antimicrobial composition comprising the isolated polypeptide according to claim 26 and an oxygen donor.

36. The composition according to claim 35, wherein the oxygen donor is peroxide or a source of hydrogen peroxide.

37. The composition according to claim 35, wherein the oxygen donor is a source of hydrogen peroxide that is a peroxide-producing oxidase.

38. The composition according to claim 35, wherein the oxygen donor is a peroxide-producing oxidase that is glucose oxidase, and the composition further comprises glucose or a source of glucose.

39. The composition according to claim 35, wherein the concentration ratio between the isolated polypeptide and a peroxide-producing oxidase as an oxygen donor is of at least about 10:1 in said composition.

40. The composition according to claim 35, further comprising one or more halides or pseudohalides.

41. A medical device treated with or coated with the isolated polypeptide according claim 26.

42. A method for halogenating a non-halogenated organic compound, said method comprising contacting said non-halogenated compound with a polypeptide according to claim 26.

43. A method for inhibiting the growth or killing microorganisms, said method comprising contacting microorganisms with a polypeptide according to claim 26.

44. A method for treating a microbial infection in a subject in need thereof, said method comprising administering to the subject a therapeutically effective amount of a polypeptide according to claim 26.

45. A method for converting hydrogen peroxide into oxygen and water, said method comprising contacting hydrogen peroxide with a polypeptide according to claim 26.


SEQUENCE LISTING (36 sequences)

SEQ ID NO: 1 - protein, 713 aa, Rhodopirellula baltica
MLFWSKRTRKHPGRKRRRLRSEQLESRQLLAADFGGYRHNIYDAEDVNDDGRVSAIDALLVINAMTSETSRDENVFTDVNGDGRRSALDALRVINRIERGDVFPIDTSGDEDLNTLLPEMPSEVRSIDGSGNNPNDHLWGSVGQRLIRMAPAAYGDGISTLAGANRPSAREVSNVLSAMNTSQVLNDRGLSAFVYVWGQFIDHDLGLTESDEHGEAIDISVPEGDLWFDPMGSGEAVIPMTRTPIADGTGTSVGNPAQQFNQITAFIDGSMVYGSDAATAERLRTFAGGRLAISDNGLLPMDESGMVIAGDVRASENVGLTAIQTLFVREHNRLADEIFAADPEATDEEIYQRARLVVASLIQSITYNEFLPALLGQRAMDSYRGYDASVNPGIANEFSTAAFRLGHSTLRDEVGFMSNDGRESKDEMELKDAFFHASMLEETGIDSLLKFDASVQAQEIDLAVVDSLRNFLFGPPGAGGLDLVAMNIQRGRDHGLSDYNTTRQAYGLDRVETFDQITGDVELQQKLASLYGTVDNIDLWVGLMAEDHQHDASVGELTGLIIADQFQRTRDGDRFFYKNVLTDSEVESIERSTLADLIERNTSVDGLQENVFFMQVELRGRVILASSIPPNTMPSEVREEEIRGVSGMAIELIDASGKLVDSTVSDQHGNYRFRALSETGDYQVRLSESGEAMDVLVAHGAERIRGLDFIVLA

SEQ ID NO: 2 - DNA, 2142 bp, Rhodopirellula baltica
  source 1..2142: mol_type = genomic DNA
atgttgttctggtcgaaacgtacgaggaagcatccaggtcgcaagcgacgtcgattgcgaagcgaacaactggaatctcgtcagctgcttgccgccgattttggcgggtatcgtcacaacatctatgacgcagaagacgtcaacgatgacggtcgtgtttcagccatcgacgcgttgttggtcatcaatgcgatgaccagtgagacctcgcgagatgaaaacgtgttcaccgatgtcaacggtgatggtcgacgcagtgctttggacgcgcttcgtgtgatcaaccgaatcgaacgcggcgatgtgttcccgatagacaccagtggcgacgaggatctgaacacgctgcttccggagatgccgtctgaggttcgttcgatcgatggctcgggaaacaatcccaacgatcatctttggggcagcgtcgggcaacgtttgatacgcatggcgccggcagcctacggtgacggcatttcgactttggcgggagcgaaccggcccagcgcacgcgaagtcagcaacgtgttgtcggcgatgaacacgagccaggtactcaatgatcgtgggctatccgcttttgtttatgtgtgggggcaattcatcgatcatgacttaggattgactgaaagcgacgaacacggcgaagcgatcgatatctcagtaccggaaggtgatctttggtttgatccgatgggttccggagaagcggtgattccgatgacgcgaacgccgatcgcggacggaactggaacttcggttggcaaccctgcgcaacagttcaatcagatcacggccttcatcgatggatcgatggtttacgggagtgatgcggcaaccgcggaaagactgaggacgtttgcgggcggtcgattggcgatctccgacaacgggttgcttccgatggacgagagcggcatggtgattgccggtgatgttcgtgccagcgagaacgtgggtttgacggcgattcagacgttgttcgtccgagagcacaatcgtttggcagacgagatttttgcagcggatcctgaggcaactgacgaagaaatttatcaacgagcccggttggtcgtcgccagtttgatccaatccattacctacaacgagttcttgccagcactgttggggcagcgtgcgatggattcgtatcgcggttacgacgcgtccgtgaatcccggcatcgcgaatgagttttcaaccgccgcgtttcggttggggcacagcacgcttcgtgatgaagttggattcatgtccaatgatggacgtgagagcaaagacgagatggaactgaaagacgcgttcttccatgcgtcgatgttggaagagaccggcattgattctctgttgaagtttgacgcgtccgtgcaggctcaggaaattgatttggccgtggtcgattcgttgcggaacttcttgtttgggcctcctggagcgggcggattggatctcgtcgcgatgaacatccaacgcggccgcgatcatggcctgagcgattacaacacaacacggcaagcgtacggtttggaccgagtggaaacgtttgaccaaatcaccggcgatgtggagttgcagcagaagttggcgtcgctgtacggcaccgtcgacaacattgatttgtgggttggtttgatggccgaggatcaccaacatgatgcatccgtcggcgaactgacgggtttgatcatcgccgatcaattccagaggactcgtgatggagatcggttcttctacaaaaacgtcttgaccgattcggaggttgagtcgattgagcgttctaccttggcagatctgattgaacgcaacacgagtgtggacggattgcaggaaaacgtcttcttcatgcaggtcgaacttcgcgggcgagtcatcttggccagctcgattccgccgaacaccatgccgagcgaggtgcgtgaagaggagattcgcggtgtgtcgggaatggcgatcgagttgattgacgcaagcgggaagctggttgattccacggtgtccgatcaacatgggaattatcgcttccgagcgttgagcgagacaggtgattatcaggttcgcttgtcggaatcgggcgaagcgatggatgtcctcgtcgctcacggtgcggaaagaattcgcggcctggattttatcgtcctcgcttga

SEQ ID NO: 3 - DNA, 36 bp, synthetic construct
  misc_feature 1..36: note = primer
  source 1..36: mol_type = other DNA
gccaacgttgttgcacatcatatgttgttctggtcg

SEQ ID NO: 4 - DNA, 33 bp, synthetic construct
  misc_feature 1..33: note = primer
  source 1..33: mol_type = other DNA
gcttctacggtgccgaactcgagagcgaggacg

SEQ ID NO: 5 - DNA, 33 bp, synthetic construct
  misc_feature 1..33: note = primer
  source 1..33: mol_type = other DNA
tttatcgtcctcgcttgagagcaccaccaccac

SEQ ID NO: 6 - DNA, 33 bp, synthetic construct
  misc_feature 1..33: note = primer
  source 1..33: mol_type = other DNA
gttcgtgccagcgagatggtgggtttgacggcg

SEQ ID NO: 7 - DNA, 33 bp, synthetic construct
  misc_feature 1..33: note = primer
  source 1..33: mol_type = other DNA
gggggcaattcatcgcgcatgacttaggattgc

SEQ ID NO: 8 - DNA, 33 bp, synthetic construct
  misc_feature 1..33: note = primer
  source 1..33: mol_type = other DNA
gttcgtgccagcgcgaacgtgggtttgacggcg

SEQ ID NO: 9 - DNA, 36 bp, synthetic construct
  misc_feature 1..36: note = primer
  source 1..36: mol_type = other DNA
ccgccgcgtttcggttgggggcgagcacgcttcgtg

SEQ ID NO: 10 - DNA, 34 bp, synthetic construct
  misc_feature 1..34: note = primer
  source 1..34: mol_type = other DNA
gggcaattcatcgatgcggacttaggattgactg

SEQ ID NO: 11 - DNA, 33 bp, synthetic construct
  misc_feature 1..33: note = primer
  source 1..33: mol_type = other DNA
gtttatgtgtggggggcgttcatcgatcatgac

SEQ ID NO: 12 - protein, 10 aa, synthetic construct
  REGION 1..10: note = RbMPO amino acids 196 to 205
VWGQFIDHDL

SEQ ID NO: 13 - protein, 9 aa, synthetic construct
  REGION 1..9: note = RbMPO amino acids 263 to 271
ITAFIDGSM

SEQ ID NO: 14 - protein, 6 aa, synthetic construct
  REGION 1..6: note = RbMPO amino acids 312 to 317
VRASEN

SEQ ID NO: 15 - protein, 8 aa, synthetic construct
  REGION 1..8: note = RbMPO amino acids 402 to 409
AFRLGHST

SEQ ID NO: 16 - protein, 4 aa, synthetic construct
  REGION 1..4: note = RbMPO amino acids 486 to 489
MNIQ

SEQ ID NO: 17 - protein, 9 aa, synthetic construct
  REGION 1..9: note = *hMPO amino acids 88 to 97
QWQLLDHDL

SEQ ID NO: 18 - protein, 9 aa, synthetic construct
  REGION 1..9: note = *hMPO amino acids 167 to 175
LTSFVDASM

SEQ ID NO: 19 - protein, 6 aa, synthetic construct
  REGION 1..6: note = *hMPO amino acids 238 to 243
TRSSEM

SEQ ID NO: 20 - protein, 8 aa, synthetic construct
  REGION 1..8: note = *hMPO amino acids 331 to 338
AFRYGHTL

SEQ ID NO: 21 - protein, 4 aa, synthetic construct
  REGION 1..4: note = *hMPO amino acids 420 to 423
LNMQ

SEQ ID NO: 22 - protein, 10 aa, synthetic construct
  REGION 1..10: note = LPO amino acids 219 to 228
QWGQIVDHDL

SEQ ID NO: 23 - protein, 9 aa, synthetic construct
  REGION 1..9: note = LPO amino acids 300 to 308
LTSFLDASF

SEQ ID NO: 24 - protein, 6 aa, synthetic construct
  REGION 1..6: note = LPO amino acids 371 to 376
SRASEH

SEQ ID NO: 25 - protein, 8 aa, synthetic construct
  REGION 1..8: note = LPO amino acids 463 to 470
AFRFGHLE

SEQ ID NO: 26 - protein, 4 aa, synthetic construct
  REGION 1..4: note = LPO amino acids 553 to 556
INTQ

SEQ ID NO: 27 - protein, 10 aa, synthetic construct
  REGION 1..10: note = EPO amino acids 226 to 235
QWGQFIDHDL

SEQ ID NO: 28 - protein, 9 aa, synthetic construct
  REGION 1..9: note = EPO amino acids 305 to 313
LTSFVDASM

SEQ ID NO: 29 - protein, 6 aa, synthetic construct
  REGION 1..6: note = EPO amino acids 377 to 382
TRSTET

SEQ ID NO: 30 - protein, 8 aa, synthetic construct
  REGION 1..8: note = EPO amino acids 469 to 476
AFRFGHTM

SEQ ID NO: 31 - protein, 4 aa, synthetic construct
  REGION 1..4: note = EPO amino acids 558 to 561
LNMQ

SEQ ID NO: 32 - protein, 10 aa, synthetic construct
  REGION 1..10: note = TPO amino acids 232 to 241
AWGQYIDHDI

SEQ ID NO: 33 - protein, 9 aa, synthetic construct
  REGION 1..9: note = TPO amino acids 320 to 328
LTSFLDAST

SEQ ID NO: 34 - protein, 6 aa, synthetic construct
  REGION 1..6: note = TPO amino acids 395 to 400
GRASEV

SEQ ID NO: 35 - protein, 8 aa, synthetic construct
  REGION 1..8: note = TPO amino acids 489 to 496
AFRFGHAT

SEQ ID NO: 36 - protein, 4 aa, synthetic construct
  REGION 1..4: note = TPO amino acids 578 to 581
INLQ